SEQUENCE LISTING (7 sequences)

SEQ ID NO: 1 - DNA, 1522 bp, Artificial sequence
  sequence encoding synthetic RNA template for VLP production
gggcgaattggagctcgaaaagaaacacaaagcaaagcaaagcaaagcaactcaaataaaccatatttggccaaggcacttggtaatcaaacgggcacaaccctagattaacgattaagcaaatttgaggagtgttttcgaacagttgaacgacgtctcactccgggctgttattcaagaagaggcctacagagacattaagctcactattaaggaaactaaaacctacaatcctttaacacatccagtagcagtagcagatagtttagaaaaattaggaatagaaactaacccctttgccgtcaaggcgcatacgctaaccgcggcaaaaacaatagaattagattaatacaaaatagtttctttctacctcccaaaggagaaccccactaccttttaattctaaaagaggagcaagttgcaatattttagaagaggcccacagcaaaaagtaatattcctcataactcacatagaacccaaagacgtggctaggttaaacgtggacaccctttttgacaagaacgtgaccccacagattaccacaaacacagccttttaaggggataccctccattttctcccactaacagcgattgaaaggatttttaaatcctcccccaacttcaaaccctctacgccacttaagtactcccaccggaggccctgcataggctgcattccctgcaccctggtatattaaaattagagtttcaccaagaacatttcatctacaaaccagggggtctaactggggcagcgtacatccacaaatacgagcaactcgagtggattaaagtgggaaggtttaagtgggcggacgggaaggggcttacccacacggtgacctcacaaattttggaaactaaaggtgccaaccacctcttcatttttcagagagggaggtttctgactccagaattgaggtgtttcaacactgagacaaaattaatcacctaacctcccatcttcctccccaagcagtttataacccggttgccaattaagaaaaccaaccggtccgcccaacaattgtttctctacgtaaaatcagtgaagataataacagagagggacatctgggcaaagtaaagacagttgctcaagacttcggagctacaagattacaatccaagggaggtggctctgctggtgaactattttctcttgatcgccagattaaggtcggaaacgtgttttgacaacgtcctcagcgggggataattcaagaaactcttcaaacccttcatcgctaagtgggagatccaaaaacacaaaatttttggaataaaggagtttgaacagttataagaagctctggataaggtggtaataacactgacctacccaacaaaaacttttgacaatcggggttgggtggttaagctggaagcgaggaggggttacgagtggttcgctgtaaagtaacacaagccgaagggtccagaactaaacttggaggagaagaaaacggatccggacgccgcatcctacgaaaaatacttgaaagccctgaacgcgt

SEQ ID NO: 2 - protein, 215 aa, Papaya mosaic virus
  wild-type PapMV coat protein
MSKSSMSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLIRSPTQEERIANATNKQVHLFQAAAQDNNFTSNSAFITKGQISGSTPTIQFLPPPE

SEQ ID NO: 3 - DNA, 648 bp, Papaya mosaic virus
  sequence encoding wild-type PapMV coat protein
atgtctaagtcaagtatgtccacacccaacatagccttccccgccatcacccaggaacagatgagctcgattaaggtcgatccaacgtccaatcttctgccctcccaagagcagttaaagtcagtgtccaccctcatggtagctgctaaggttccagcagccagtgttacaactgtggcattggagttggtcaacttctgctatgacaatgggtccagcgcgtacaccacagtgactggcccatcatcaataccggagatatcactggcacaattggctagtattgtcaaagcttccggcacttcccttagaaaattctgccggtacttcgcgccaataatctggaatctgaggacggacaaaatggctcctgccaattgggaggcttcaggatacaagccaagcgccaaatttgccgcgttcgacttcttcgacggggtggagaatccggcggccatgcaacccccttcgggactaatcaggtcgccgacccaggaagagcggattgccaatgctaccaacaaacaggtgcatctcttccaagccgcggcacaggacaacaactttaccagcaactccgccttcatcaccaaaggccaaatttctgggtcaaccccaaccatccaattccttccaccccccgaataa

SEQ ID NO: 4 - protein, 211 aa, Artificial sequence
  modified PapMV coat protein CPdeltaN5
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPE

SEQ ID NO: 5 - protein, 222 aa, Artificial sequence
  modified PapMV coat protein PapMV CPsm
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSTTRHHHHHH

SEQ ID NO: 6 - DNA, 2662 bp, Artificial sequence
  sequence encoding synthetic RNA template for VLP production
gggcgaattggagctcgaaaagaaacacaaagcaaagcaaagcaaagcaactcaaataaaccatatttggccaaggcacttggtaatcaaacgggcacaaccctagattaacgattaagcaaatttgaggagtgttttcgaacagttgaacgacgtctcactccgggctgttattcaagaagaggcctacagagacattaagctcactattaaggaaactaaaacctacaatcctttaacacatccagtagcagtagcagatagtttagaaaaattaggaatagaaactaacccctttgccgtcaaggcgcatacgctaaccgcggcaaaaacaatagaattagattaatacaaaatagtttctttctacctcccaaaggagaaccccactaccttttaattctaaaagaggagcaagttgcaatattttagaagaggcccacagcaaaaagtaatattcctcataactcacatagaacccaaagacgtggctaggttaaacgtggacaccctttttgacaagaacgtgaccccacagattaccacaaacacagccttttaaggggataccctccattttctcccactaacagcgattgaaaggatttttaaatcctcccccaacttcaaaccctctacgccacttaagtactcccaccggaggccctgcataggctgcattccctgcaccctggtatattaaaattagagtttcaccaagaacatttcatctacaaaccagggggtctaactggggcagcgtacatccacaaatacgagcaactcgagtggattaaagtgggaaggtttaagtgggcggacgggaaggggcttacccacacggtgacctcacaaattttggaaactaaaggtgccaaccacctcttcatttttcagagagggaggtttctgactccagaattgaggtgtttcaacactgagacaaaattaatcacctaacctcccatcttcctccccaagcagtttataacccggttgccaattaagaaaaccaaccggtccgcccaacaattgtttctctacgtaaaatcagtgaagataataacagagagggacatctgggcaaagtaaagacagttgctcaagacttcggagctacaagattacaatccaagggaggtggctctgctggtgaactattttctcttgatcgccagattaaggtcggaaacgtgttttgacaacgtcctcagcgggggataattcaagaaactcttcaaacccttcatcgctaagtgggagatccaaaaacacaaaatttttggaataaaggagtttgaacagttataagaagctctggataaggtggtaataacactgacctacccaacaaaaacttttgacaatcggggttgggtggttaagctggaagcgaggaggggttacgagtggttcgctgtaaagtaacacaagccgaagggtccagaactaaacttggaggagaagaaaacggatccggacgccgcatcctacgaaaaatacttgaaagccctgaacgcgtgccttttgcagaaagaaccggaggtctaagaagccaaagaagcagaggcaacagtaaaaccccaaaggcccgaaattaaggaagagcaggcggaggcttccacgagtgggagggctgaagaaattcaagaggatccggcaaccaggaaagggaaggaggagccgaaccccaatcgggacctgctctgccctaacggattacatctaaagatcaaaataaccgaatttcctgaacttccggtactcgatcatccagatcatctcaccggaagaaaagctaagttcttttcaaaggtaagaaagccatactcctacacaggaggctcactaacatctcgaggctggccaaattggctggagaaaattttagctgcaatagaaatcaaagagccactgccggaattcaaccataacttagtccagcagtttaaactccaagcggccatcccattccaccgagtaataaaaccgtgttatccaaagggtcaccaggttggcgccctcaccatcaaccactcgggagataacctcacccaaatagcttgccaaaaaggaaaggcaagtataacctaaggattcggggactactacttgagcccagtgggattccaagagtcccacaagctaacggtgagcaacaccacagggggaagggtgtctctgaccttcagtaacacagtccagcaaaacaaatttataataataagaagttaagaagcccttaataaccttccttggaaagcgtggatcccgaaactgcaaaatttaggattccaaggacggcagcttcagttaaatcctataagagcactgattagccccactgaggaaattcgaagctaacccaagtgtaagcctgaaggggttccagaagtagtttacaagacgctggacggactagctcgtgcgccaactccatattcgccaaaccctatccgagctagagcatacacctcagtaatcaaaaactgcagaattggggccctgctaaggcaacaaggtaaggagtggggtaacaggtttgtaacattggtagaagctggcaagagagagttggccatccccgtaattcacggagcgggaggaagtgggaagtcacaagcattgcagaccctgattaaggacaacccagagcttgata

SEQ ID NO: 7 - protein, 8 aa, Artificial sequence
  OVA peptide
SIINFEKL

(19) United States
(12) Patent Application Publication
(10) Pub. No.: US 2016/0228541 A1
(43) Pub. Date: Aug. 11, 2016

(54) PAPAYA MOSAIC VIRUS AND VIRUS-LIKE PARTICLES IN CANCER THERAPY

(71) Applicants: Alain LAMARRE, Laval (CA); FOLIA BIOTECH INC., Québec (CA)

(72) Inventors: Alain LAMARRE, Laval (CA); Denis LECLERC, Levis (CA)

(21) Appl. No.: 15/022,907

(22) PCT Filed: Sep. 19, 2014
(86) PCT No.: PCT/CA2014/050908

§ 371 Date: Mar. 17, 2016

Related U.S. Application Data
(60) Provisional application No. 61/880,156, filed on Sep. 19, 2013.
(60) Provisional application No. 61/886,481, filed on Oct. 3, 2013.

(51) Int. Cl. A61K 39/39; A61K 39/00; C12N 7/00

(52) U.S. Cl. CPC A61K 39/39; C12N 7/00; A61K 39/0011; C12N 2770/40021; C12N 2770/40023; C12N 2770/40033; C12N 2770/40042; C12N 2770/40034; A61K 2039/525; A61K 2039/5258; A61K 2039/5154; A61K 2039/572

(57) ABSTRACT

Papaya mosaic virus and virus-like particles (VLPs) comprising ssRNA for use to inhibit cancer growth and metastasis. The PapMV and PapMV VLPs may be used alone or in combination with another cancer therapy, such as a chemotherapeutic, immunotherapeutic, or radiotherapy.

### FIELD OF THE INVENTION

The present invention relates to the field of cancer therapeutics and, in particular, to the use of papaya mosaic virus (PapMV) and virus-like particles (VLPs) in cancer therapy.

### BACKGROUND OF THE INVENTION

The immune system is known to play an important role in cancer and in the response of tumours to conventional therapeutic modalities. Immunotherapeutic approaches for the treatment of cancer have been, and are still being, developed. Passive immunotherapy with monoclonal antibodies is an important approach, however, patients undergoing passive immunotherapy frequently relapse and show a progressive decrease in response to treatment. Alternative approaches that stimulate a patient's own immune system to fight the disease are, therefore, being developed, including cancer vaccines (such as Provenge®) and non-specific immunotherapies (such as the small molecule compound imiquimod).

Imiquimod (Aldara®) is a Toll-like receptor 7 (TLR7) agonist and a powerful immunomodulator that has been approved in the form of a 5% cream formulation for the topical treatment of premalignant and early skin cancers. Systemic administration of a similar imidazoquinoline small molecule, 852A, which is also a TLR7 agonist, was shown to result in prolonged disease stabilization in some patients with stage IV metastatic melanoma (Dudek et al., 2007, Clin Cancer Res, 13(23):7119-7125). Systemic administration of another imidazoquinoline TLR7 agonist, R848 (Resquimod), in combination with radiotherapy has been shown to lead to longstanding clearance of tumour in T- and B-cell lymphoma bearing mice (Dovedi et al., 2012, Blood, 121(2):251-259). Combination of topically applied imiquimod with local radiotherapy and systemic administration of cyclophosphamide has been shown to act synergistically in reducing tumour growth and recurrence in a mouse model of cutaneous breast cancer (Dewan et al., 2012, Clin Cancer Res, 18(24):6668-6678).

The ability of papaya mosaic virus (PapMV) and PapMV virus-like particles (VLPs) to enhance the immunogenicity of antigens has been described (U.S. Pat. Nos. 7,641,896 and 8,101,189, Canadian Patent Application No. 2,434,000, and International Patent Application No. PCT/CA03/00985 (WO 2004/004761)).

In addition, International Patent Application Publication No. WO 2012/155261 describes use of compositions comprising PapMV or PapMV VLPs for stimulation of the innate immune response. The PapMV compositions can be used to provide protection against subsequent pathogen challenge or to treat an established infection. The use of PapMV compositions to protect a subject from potential infection by a pathogen, and administration of the compositions via intranasal or pulmonary routes to elicit effects within the mucosa and/or in the respiratory system are also described.

International Patent Application Publication No. WO 2012/155262 describes an in vitro process for preparing VLPs from recombinant papaya mosaic virus coat protein and ssRNA. The VLPs can be used as adjuvants and when fused to an antigen, as vaccines. The use of the VLPs for stimulation of the innate immune response is also described.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to papaya mosaic virus and virus-like particles in cancer therapy. In one aspect, the invention relates to a composition comprising papaya mosaic virus (PapMV) or PapMV virus-like particles (VLPs) comprising ssRNA for use in the treatment of cancer in a subject in need thereof

In another aspect, the invention relates to a composition comprising papaya mosaic virus (PapMV) or PapMV virus-like particles (VLPs) comprising ssRNA for use to improve a cancer immunotherapy in treatment of cancer in a subject in need thereof.

In another aspect, the invention relates to a method of treating cancer in a subject comprising administering to the subject an effective amount of a composition comprising papaya mosaic virus (PapMV) or PapMV virus-like particles (VLPs) comprising ssRNA.

In another aspect, the invention relates to a method of improving a cancer immunotherapy in treating cancer in a subject comprising administering to the subject an effective amount of a composition comprising papaya mosaic virus (PapMV) or PapMV virus-like particles (VLPs) comprising ssRNA.

In certain embodiments, the composition comprises PapMV VLPs comprising ssRNA.

In another aspect, the invention relates to a composition comprising papaya mosaic virus (PapMV) virus-like particles (VLPs) comprising ssRNA for use in the treatment of cancer in a subject in need thereof, wherein the composition is for intratumoral administration and wherein the composition inhibits growth of the cancer.

In another aspect, the invention relates to a composition comprising papaya mosaic virus (PapMV) virus-like particles (VLPs) comprising ssRNA for use to improve a dendritic cell-based immunotherapy in treatment of cancer in a subject in need thereof.

In another aspect, the invention relates to a method of treating cancer in a subject comprising administering to the subject an effective amount of a composition comprising papaya mosaic virus (PapMV) virus-like particles (VLPs) comprising ssRNA, wherein the composition is administered intratumorally and wherein the composition inhibits growth of the cancer.

In another aspect, the invention relates to a method of improving a dendritic cell-based immunotherapy in treating cancer in a subject comprising administering to the subject an effective amount of a composition comprising papaya mosaic virus (PapMV) virus-like particles (VLPs) comprising ssRNA.

In certain embodiments, the cancer therapy comprises one or more of radiotherapy, chemotherapy and immunotherapy. In some embodiments, the cancer therapy comprises an immunotherapeutic, such as a cell-based cancer immunotherapeutic. In some embodiments, the cancer therapy comprises a dendritic cell-based immunotherapeutic.

### BRIEF DESCRIPTION OF THE DRAWINGS

These and other features of the invention will become more apparent in the following detailed description in which reference is made to the appended drawings.

FIG. 1 presents (A) the sequence of a synthetic RNA template (SRT) (SEQ ID NO:1) that can be used to prepare the PapMV VLPs in one embodiment of the invention, and (B) the sequence of another synthetic RNA template (SRT) (SEQ ID NO:6) that can be used in another embodiment of the invention; all ATG codons have been mutated for TAA stop codons (bold), the first 16 nucleotides are from the T7 transcription start site located within the pBluescript expression vector and the sequence comprises the PapMV nucleation site for rVLP assembly (boxed in (A)).

FIG. 2 presents (A) the amino acid sequence of the wild-type PapMV coat protein (SEQ ID NO:2) and (B) the nucleotide sequence of the wild-type PapMV coat protein (SEQ ID NO:3).

FIG. 3 presents (A) the amino acid sequence of the modified PapMV coat protein CPΔN5 (SEQ ID NO:4), and (B) the amino acid sequence of modified PapMV coat protein PapMV CPsm (SEQ ID NO:5).

FIGS. 4A & 4B presents graphs showing that immunization with PapMV ssRNA-VLPs intra-tumorally results in production of IFN-α 6 h post immunization. The kinetics of IFN-α in the tumour (A) and the spleen (B) were measured by ELISA.

FIGS. 4C & 4D presents graphs showing that immunization with PapMV ssRNA-VLPs induces immune cell infiltration into the tumour: (C) Flow cytometry analysis of the proportion of CD45⁺ cells, and (D) proportion of CD8⁺ and CD4⁻ T cells, B lymphocytes and plasmacytoid dendritic cells, in the tumour 24 h post-immunization.

FIG. 5 presents graphs showing that sub-cutaneous (s.c.) and intravenous (i.v.) injections of OVA-loaded BMDC (BMDC-OVA) induce the production of OVA specific CD8⁺ T cells mostly in the spleen and sera: Flow cytometry analysis of Kb-OVA⁺ cells in CD8⁺ T cells in the spleen (A), the serum (B) and lymph (C) 7 days post BMDC-OVA immunization.

FIGS. 6A, 6B & 6C presents graphs showing that intra-tumoral injection of PapMV ssRNA-VLPs increases the therapeutic effect of BMDC-OVA immunization: Growth kinetics of (A) B16-OVA-ofl and (B) B16-OVA are shown. Tumours were measured using a caliper and treatments were administered at day 7 and 12 post-inoculation of tumour cells. (C) Proportion of CD44+Kb-OVA+CD8+ T cell in the spleen at day 16 post-inoculation.

FIG. 6D presents a graph showing that complement depletion in mice does not improve the therapeutic effect of intra-tumoral treatment with PapMV ssRNA-VLPs on the growth kinetics of sub-cutaneous melanoma B16-OVA (measured by caliper following treatment at days 7 and 12).

FIG. 7 presents graphs showing that complement depletion did not induce a significant generation of OVA-specific CD8⁺ T cells in the lung of B16-OVA i.v. inoculated mice: Flow cytometry analysis of Kb-OVA specific CD8⁺ T cells (A), and IL-2 producing CD8⁺ T cells (B), in the lung 7 days post-immunization.

FIG. 8 presents graphs showing that pretreatment with PapMV ssRNA-VLPs increased the therapeutic effect of BMDC-OVA immunization on B16-OVA metastasis. Mice were inoculated i.v. with B16-OVA-ofl and PapMV ssRNA-VLPs+BMDC-OVA were injected at day 7 post-inoculation. Mice were sacrificed at day 12 and the lungs were harvested. (A) Luciferin was added to the lung homogenate supernatant and luminescence has measured using luminometer. Proportion of CD44⁺Kb-OVA⁺CD8⁺ T cell in the lung (B) and the spleen (C). Proportion of splenic CD8⁺ T cell producing IFN-γ (D) or TNF-α (E) following in vitro restimulation with the OVA peptide SIINFEKL (SEQ ID NO:7).

FIG. 9 presents results showing that treatment with PapMV ssRNA-VLPs decreases the growth rate of B16-OVA melanoma and increases immune cell infiltration: (A) Tumour growth was followed with the measure of the tumour diameter using a caliper and calculation of the tumour area. (B) Immune cell infiltration was determined by flow cytometry with the proportion of CD45+ cells in the tumour. (C) Proportion of CD44+Kb-OVA+CD8+ T cell in the CD45+ population of the tumour homogenate. (D) Proportion of CD8+ T cell in the tumour producing IFN-γ following in vitro restimulation with OVA peptide SIINFEKL (SEQ ID NO:7). *:P<0.05

FIG. 10 presents graphs indicating the presence of (A) MIP-1α, (B) MIP-1β, (C) MIP-2, (D) KC, (E) TNF-α, (F) RANTES, (G) VEGF, (H) MCP-1, (I) IP-10, (J) IL-17, (K) IL-13, (L) IL-12 (p70), (M) IL-9, (N) IL-6, (O) IL-1α, (P) IL-1β, (Q) GM-CSF and (R) G-CSF in bronchoalveolar lavage of Balb/C mice treated intranasally with one or two treatments of PapMV ssRNA-VLPs (60 μg) or with control buffer (Tris HCl 10 mM pH 8). Each point corresponds to the level of cytokines detected in each mouse.

FIG. 11 presents graphs depicting evaluation by ELISA of the kinetics of production of IFN-α in serum (A) and spleen (B) of C57BL/6 mice following intra-venous immunization with 100 μg PapMV ssRNA-VLPs; and (C) ELISA quantification of serum IFN-α in C57BL/6 and different knockout mice 6 h post-immunization (i.v.) with 100 μg PapMV ssRNA-VLPs or PBS.

FIG. 12 presents results showing that intra-peritoneal administration of PapMV ssRNA-VLPs induces production of cytokines and chemokines in the spleen of mice, (A) IFN-gamma (IFN-g), (B) IL-6, (C) TNF-alpha (TNF-α), (D) KC and (E) the chemokine MIP-1alpha (MIP-1a).

FIG. 13 presents results showing that intra-peritoneal administration of PapMV ssRNA-VLPs induces production of cytokines and chemokines in the serum of mice, (A) KC, (B) IFN-gamma (IFN-g), (C) IL-6, (D) the chemokine MIP-1 alpha (MIP-1a), and (E) TNF-alpha (TNF-a).

FIG. 14 presents results showing that intra-peritoneal administration of PapMV ssRNA-VLPs induces production of IFN-alpha (IFN-a) in the spleen (A) and the serum (B) of mice, and also induces secretion of KC (C) and MIP1-a (D) in the serum of the mice 5 hours after treatment (PolyC=PapMV VLPs self-assembled with PolyC DNA; PapMV and ENG=PapMV ssRNA-VLPs; Dénat=denatured PapMV ssRNA-VLPs; 5715=PapMV VLP batch with weak adjuvant activity).

FIG. 15 presents results showing that PapMV ssRNA-VLPs treatment decreases the growth rate of B16-OVA melanoma and increases immune cell infiltration. (A) Tumour growth was followed by measurement of the tumour diameter with calipers and calculation of the tumor area (mm²) (B) Percentage survival of mice. Mice were euthanized when the tumour reached a diameter of 17 mm Luminex quantification of (C) IP-10 (D) MCP-1 and (E) IL-6 in the tumour 6 h post-injection of PapMV ssRNA-VLPs. (F) Immune cell infiltration was determined by flow cytometry with the proportion of CD45⁺ cells in the tumour. Proportion of (G) CD8⁺ T cells, (H) myeloid-derived suppressor cells (MDSC, CD11b^{hi}Gr1⁺) (I) Kb-OVA⁺CD8⁺ T cell, (J) Db-gp100⁺CD8⁺ T cell and (K) Kb-TRP2⁺CD8⁺ T cell in the CD45⁺ population of the tumor homogenate at day 15 post-inoculation. *:P<0.05, ***:P<0.001.

FIG. 16 presents results showing that pretreatment with PapMV ssRNA-VLPs increases the therapeutic effect of DC-OVA immunization on B16-OVA melanoma tumour. (A) Tumour growth was monitored over time using calipers. (B) Percentage survival of mice. Mice were euthanized when the tumour reached a diameter of 17 mm *: p<0.05.

FIG. 17 presents results showing the effect of PapMV ssRNA-VLPs in combination with high dose cyclophosphamide (CTX; 100 mg/kg) on tumour growth. (A) PapMV ssRNA-VLPs administered intravenously, and (B) PapMV ssRNA-VLPs administered intratumorally.

### DETAILED DESCRIPTION OF THE INVENTION

The present invention relates generally to the use of Papaya Mosaic Virus

(PapMV) and PapMV virus-like particles (VLPs) comprising ssRNA (ssRNA-VLPs) in cancer therapy and is based on the finding that, in addition to their ability to act as adjuvants in enhancing a newly triggered immune response against an antigen, PapMV and PapMV ssRNA-VLPs are capable of potentiating existing immune responses in subjects with cancer to a level sufficient to provide an anti-cancer effect.

As shown herein, PapMV ssRNA-VLPs are capable of inhibiting tumour growth when administered alone, and are also capable enhancing the tumour growth reduction effects and/or anti-metastatic effects of other cancer therapies, and in particular cancer immunotherapies. Without being limited to any particular theory, it is believed that PapMV and PapMV ssRNA-VLPs activate the toll-like receptor, TLR7, enabling them to act as immunomodulators and potentiate the activity of a patient's immune cells against a tumour. As PapMV also contains endogenous ssRNA, it is predicted to exhibit analogous immunomodulatory effects against tumours.

Accordingly, in certain embodiments, the invention relates to methods of using PapMV and PapMV ssRNA-VLPs as immunomodulators in cancer therapy. Some embodiments relate to methods of using the PapMV or PapMV ssRNA-VLPs alone to inhibit the growth of a tumour.

The use of PapMV and PapMV ssRNA-VLPs to boost the anti-cancer immune response in a patient undergoing another cancer therapy and thus improve the effectiveness of the therapy is also contemplated in certain embodiments. Some embodiments of the invention thus relate to methods of using PapMV or PapMV ssRNA-VLPs as part of a combination therapy to treat cancer, for example, to inhibit growth of a tumour and/or to inhibit metastasis of a tumour. Combination therapies contemplated in various embodiments of the invention include, for example, combination of the PapMV or PapMV ssRNA-VLPs with one or more of an immunotherapeutic, a chemotherapeutic, radiotherapy or virotherapy.

Some embodiments of the invention thus relate to therapeutic combinations that comprise the PapMV or PapMV ssRNA-VLPs and another cancer therapeutic, for example, an immunotherapeutic or a chemotherapeutic.

In some embodiments, it is contemplated that the PapMV or PapMV ssRNA-VLPs may be administered in combination with a therapeutic cancer vaccine or other cancer immunotherapeutic to inhibit tumour growth or metastasis. In some embodiments, it is contemplated that the PapMV or PapMV ssRNA-VLPs may be administered in combination with a cancer immunotherapeutic to inhibit tumour growth or metastasis. In certain embodiments, the PapMV or PapMV ssRNA-VLPs may be administered in combination with a cell-based cancer immunotherapeutic, such as a dendritic cell (DC)-based cancer immunotherapeutic. Certain embodiments of the invention relate to methods of using PapMV or PapMV ssRNA-VLPs in combination with one or more therapeutic cancer vaccines or other cancer immunotherapeutics to inhibit metastasis of a tumour.

Certain embodiments of the invention relate to methods of using PapMV or PapMV ssRNA-VLPs to improve an immunotherapy comprising dendritic cells loaded with a cancer specific antigen. In this context, the PapMV or PapMV ssRNA-VLPs may be used, for example, as a pretreatment before the administration of the antigen-loaded dendritic cells in order to improve the efficacy of the dendritic cell treatment through stimulation of the innate immunity of the patient prior to administration of the loaded dendritic cells, or the PapMV or PapMV ssRNA-VLPs may be administered concurrently with or subsequent to the antigen-loaded dendritic cells.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

“Injection” or “administration” of the PapMV or ssRNA-VLPs is intended to encompass any technique effective to introduce PapMV or ssRNA-VLPs into the body of the subject. In certain embodiments, the PapMV or ssRNA-VLPs are introduced into the body of the subject by subcutaneous, intratumoral, intraperitoneal, intravenous, intranasal or intramuscular administration.

Administration of the PapMV or ssRNA-VLPs “in combination with” one or more further therapeutic agents is intended to include simultaneous (concurrent) administration and consecutive administration. Simultaneous administration may in certain cases involve pre-mixing the PapMV or ssRNA-VLPs and the therapeutic agent(s). In some cases, simultaneous administration may involve concurrent administration of the PapMV or ssRNA-VLPs and the therapeutic agent(s) without pre-mixing. Consecutive administration is intended to encompass various orders of administration of the PapMV or ssRNA-VLPs and therapeutic agent(s) to a subject with administration of the PapMV or ssRNA-VLPs and therapeutic agent(s) being separated by a defined time period that may be short (for example in the order of minutes or even seconds) or extended (for example in the order of hours, days or weeks).

The term “inhibit” and grammatical variations thereof, as used herein, refer to a measurable decrease in a given parameter or event.

The terms “therapy” and “treatment,” as used interchangeably herein, refer to an intervention performed with the intention of alleviating the symptoms associated with, preventing or delaying the development of, or altering the pathology of, a disease or associated symptom(s). Thus, the terms therapy and treatment are used broadly, and in various embodiments include one or more of the prevention (prophylaxis), moderation, reduction, and/or curing of a disease or associated symptom(s) at various stages.

The terms “subject” and “patient” as used herein refer to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep and other hoofed animals; dogs; cats; chickens; ducks; non-human primates; guinea pigs; rabbits; ferrets; rats; hamsters and mice.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method or composition of the invention, and vice versa. Furthermore, compositions and kits of the invention can be used to achieve methods of the invention.

### Papaya Mosaic Virus and Virus-Like Particles

### PapMV

PapMV is known in the art and can be obtained, for example, from the American Type Culture Collection (ATCC) as ATCC No. PV-204™. The virus can be maintained on, and purified from, host plants such as papaya (Carica papaya) and snapdragon (Antirrhinum majus) following standard protocols (see, for example, Erickson, J. W. & Bancroft, J. B., 1978, Virology 90:36-46).

PapMV ssRNA-VLPs

PapMV ssRNA-VLPs comprise a plurality of PapMV coat proteins assembled around a ssRNA molecule to form a virus-like particle.

### PapMV Coat Protein

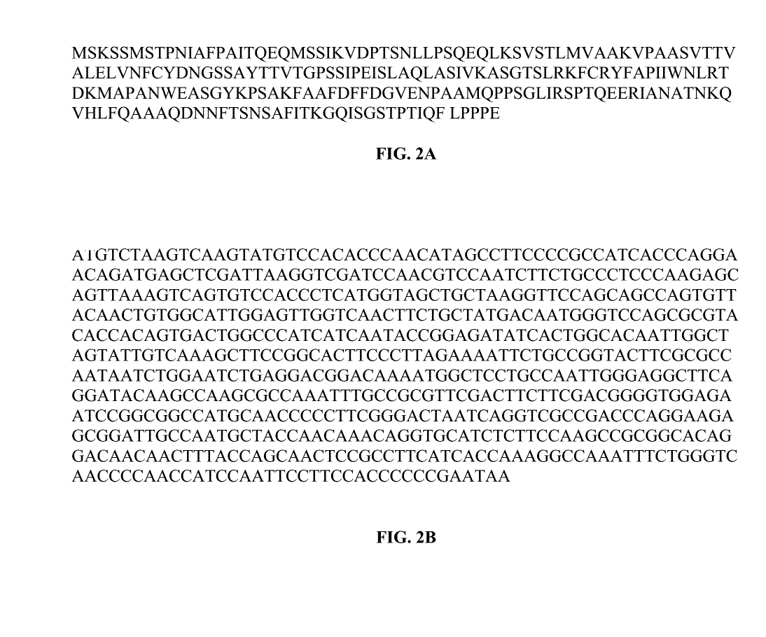

The PapMV coat protein used to prepare the VLPs can be the entire PapMV coat protein, or part thereof, or it can be a genetically modified version of the wild-type PapMV coat protein, for example, comprising one or more amino acid deletions, insertions, replacements and the like, provided that the coat protein retains the ability to self-assemble into a VLP. The amino acid sequence of the wild-type PapMV coat (or capsid) protein is known in the art (see, Sit, et al., 1989, J. Gen. Virol., 70:2325-2331, and GenBank Accession No. NP_044334.1) and is provided herein as SEQ ID NO:2 (see FIG. 2A). Variants of this sequence are known, for example, the sequences of coat proteins of Mexican isolates of PapMV described by Noa-Carrazana & Silva-Rosales (2001, Plant Science, 85:558) have 88% identity with SEQ ID NO:2 and are available from GenBank. The nucleotide sequence of the PapMV coat protein is also known in the art (see, Sit, et al., ibid., and GenBank Accession No. NC_001748 (nucleotides 5889-6536)) (see FIG. 2B; SEQ ID NO:3).

As noted above, the amino acid sequence of the PapMV coat protein need not correspond precisely to the parental (wild-type) sequence, i.e. it may be a “variant sequence.” For example, the PapMV coat protein may be mutagenized by substitution, insertion or deletion of one or more amino acid residues so that the residue at that site does not correspond to the parental (reference) sequence. One skilled in the art will appreciate, however, that such mutations will not be extensive and will not dramatically affect the ability of the recombinant PapMV CP to assemble into VLPs.

Recombinant PapMV CPs prepared using fragments of the wild-type coat protein that retain the ability to multimerise and assemble into a VLP (i.e. are “functional” fragments) are, therefore, also contemplated by the present invention for preparation of the ssRNA-VLPs. For example, a fragment may comprise a deletion of one or more amino acids from the N-terminus, the C-terminus, or the interior of the protein, or a combination thereof In general, functional fragments are at least 100 amino acids in length, for example, at least 150 amino acids, at least 160 amino acids, at least 170 amino acids, at least 180 amino acids, or at least 190 amino acids in length. Deletions made at the N-terminus of the wild-type protein should generally delete fewer than 13 amino acids, for example 12, 11, 10, 9, 8,7, 6, 5, 4, 3, 2 or 1 amino acid, in order to retain the ability of the protein to self-assemble.

In certain embodiments, when a recombinant coat protein comprises a variant sequence, the variant sequence is at least about 70% identical to the reference sequence, for example, at least about 75%, at least about 80%, at least about 85%, at least about 90%, at least about 95%, at least about 97% identical, at least about 98% identical or at least about 99% identical to the reference sequence, or any amount therebetween. In certain embodiments, the reference amino acid sequence is SEQ ID NO:2.

In certain embodiments, the PapMV coat protein used to prepare the recombinant PapMV VLPs is a genetically modified (i.e. variant) version of the

PapMV coat protein. In some embodiments, the PapMV coat protein has been genetically modified to delete amino acids from the N- or C-terminus of the protein and/or to include one or more amino acid substitutions. In some embodiments, the PapMV coat protein has been genetically modified to delete between about 1 and about 10 amino acids from the N- or C-terminus of the protein, for example, between about 1 and about 5 amino acids.

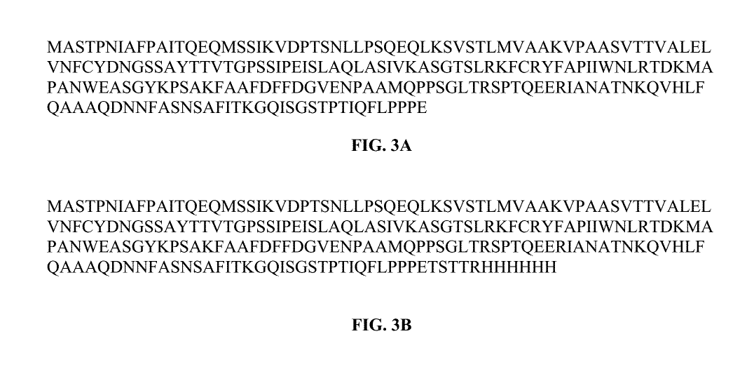

In certain embodiments, the PapMV coat protein has been genetically modified to remove one of the two methionine codons that occur proximal to the N-terminus of the wild-type protein (i.e. at positions 1 and 6 of SEQ ID NO:2) and can initiate translation. Removal of one of the translation initiation codons allows a homogeneous population of proteins to be produced. The selected methionine codon can be removed, for example, by substituting one or more of the nucleotides that make up the codon such that the codon codes for an amino acid other than methionine, or becomes a nonsense codon. Alternatively all or part of the codon, or the 5′ region of the nucleic acid encoding the protein that includes the selected codon, can be deleted. In some embodiments of the present invention, the PapMV coat protein has been genetically modified to delete between 1 and 5 amino acids from the N-terminus of the protein. In some embodiments, the genetically modified PapMV coat protein has an amino acid sequence substantially identical to SEQ ID NO:4 (FIG. 3A) and may optionally comprise a histidine tag of up to 6 histidine residues. In some embodiments, the PapMV coat protein has been genetically modified to include additional amino acids (for example between about 1 and about 8 amino acids) at the C-terminus that result from the inclusion of one or more specific restriction enzyme sites into the encoding nucleotide sequence. In some embodiments, the PapMV coat protein has an amino acid sequence substantially identical to SEQ ID NO:5 (FIG. 3B) with or without the histidine tag.

When the PapMV VLPs are prepared using a variant PapMV coat protein sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

In certain embodiments, the PapMV coat protein variant sequence comprises one or more non-conservative substitutions. Replacement of one amino acid with another having different properties may improve the properties of the coat protein. For example, as previously described, mutation of residue 128 of the coat protein improves assembly of the protein into VLPs (Tremblay et al. 2006, FEBS J., 273:14-25). In some embodiments of the present invention, therefore, the coat protein comprises a mutation at residue 128 of the coat protein in which the glutamic acid residue at this position is substituted with a neutral residue. In some embodiments, the glutamic acid residue at position 128 is substituted with an alanine residue.

Substitution of the phenylalanine residue at position F13 of the wild-type PapMV coat protein with another hydrophobic residue has been shown to result in a higher proportion of VLPs being formed when the recombinant protein is expressed than when the wild-type protein sequence is used (Laliberté-Gagné, et al., 2008, FEBS J., 275:1474-1484). In the context of the present invention, the following amino acid residues are considered to be hydrophobic residues suitable for substitution at the F13 position: Ile, Trp, Leu, Val, Met and Tyr. In some embodiments of the invention, the coat protein comprises a substitution of Phe at position 13 with Ile, Trp, Leu, Val, Met or Tyr. In some embodiments, the coat protein comprises a substitution of Phe at position 13 with Leu or Tyr.

In certain embodiments, mutation at position F13 of the coat protein may be combined with a mutation at position E128, a deletion at the N-terminus, or a combination thereof.

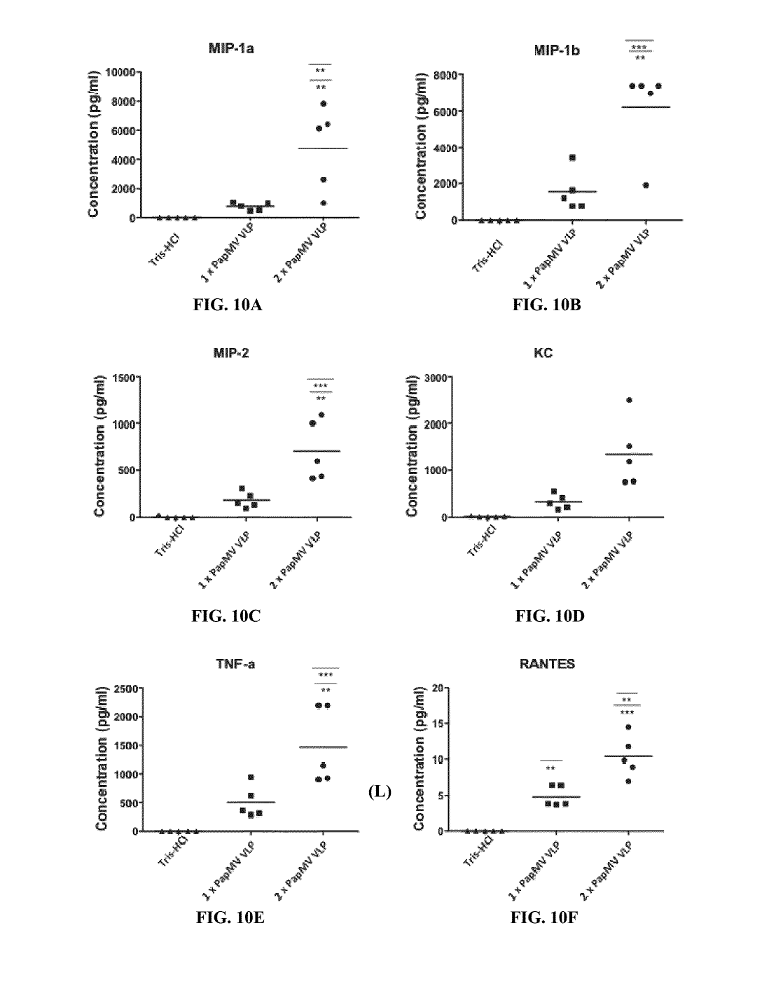

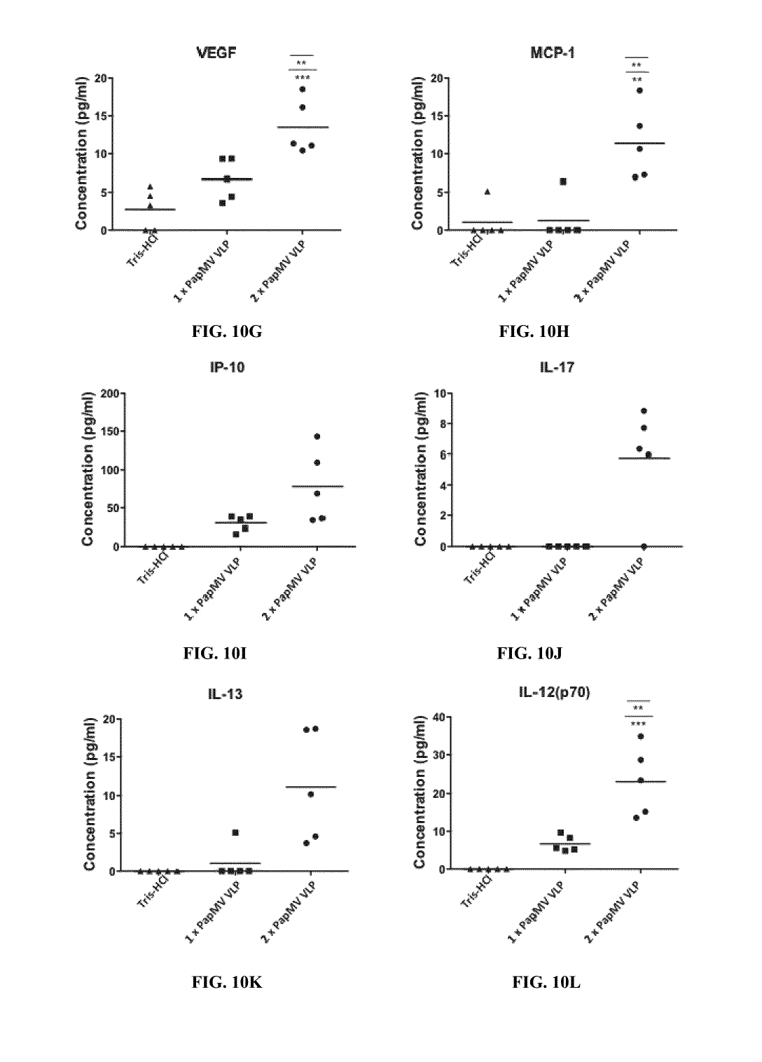

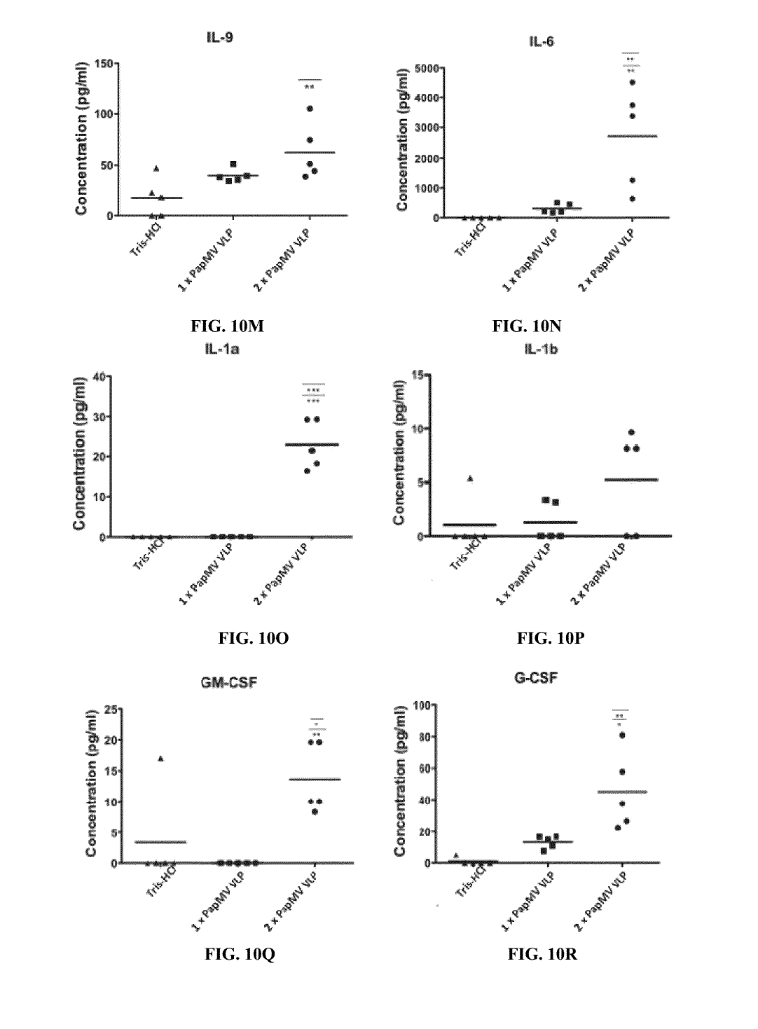

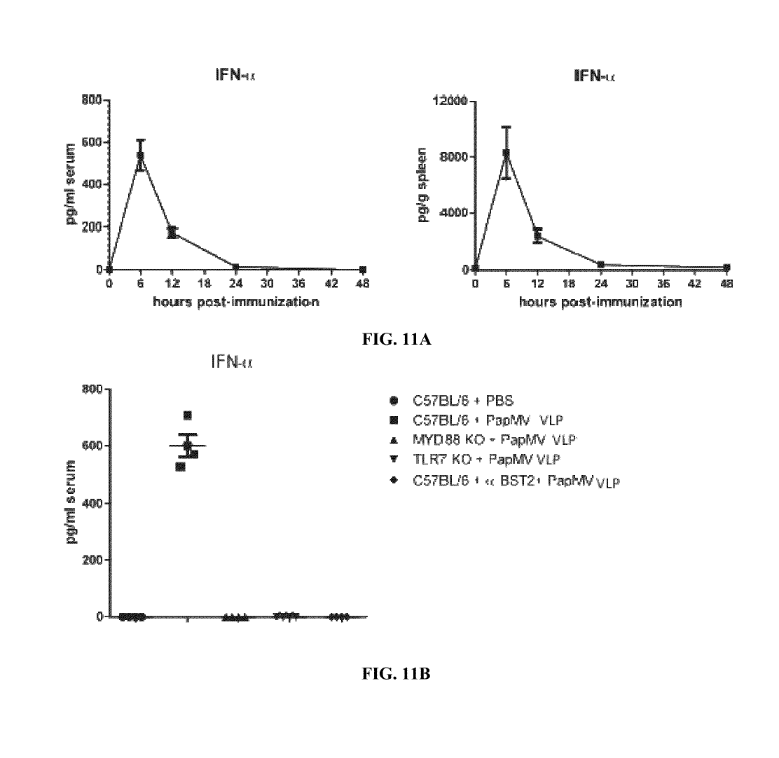

Likewise, the nucleic acid sequence encoding the PapMV coat protein used to prepare the recombinant PapMV coat protein need not correspond precisely to the parental reference sequence but may vary by virtue of the degeneracy of the genetic code and/or such that it encodes a variant amino acid sequence as described above. In certain embodiments of the present invention, therefore, the nucleic acid sequence encoding the variant coat protein is at least about 70% identical to the reference sequence, for example, at least about 75%, at least about 80%, at least about 85% or at least about 90% identical to the reference sequence, or any amount therebetween. In certain embodiments, the reference nucleic acid sequence is SEQ ID NO:3 (FIG. 10B).

### Preparation of Recombinant Coat Protein

Recombinant PapMV coat proteins for the preparation of PapMV VLPs can be readily prepared by standard genetic engineering techniques by the skilled worker. Methods of genetically engineering proteins are well known in the art (see, for example, Ausubel et al. (1994 & updates) Current Protocols in Molecular Biology, John Wiley & Sons, New York), as is the sequence of the wild-type PapMV coat protein (see, for example, SEQ ID NOs:2 and 3).

For example, isolation and cloning of the nucleic acid sequence encoding the wild-type protein can be achieved using standard techniques (see, for example, Ausubel et al., ibid.). For instance, the nucleic acid sequence can be obtained directly from the PapMV by extracting RNA by standard techniques and then synthesizing cDNA from the RNA template (for example, by RT-PCR). PapMV can be purified from infected plant leaves that show mosaic symptoms by standard techniques.

The nucleic acid sequence encoding the coat protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described previously (see for example, Tremblay, et al., 2006, ibid). In general the vector and corresponding host cell are selected such that the recombinant coat protein is expressed in the cells as low molecular weight species and not as VLPs. Selection of appropriate vector and host cell combinations in this regard is well within the ordinary skills of a worker in the art.

Alternatively, the nucleic acid sequence encoding the coat protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved, for example, by PCR-based techniques for which primers are designed that incorporate one or more nucleotide mismatches, insertions or deletions. The presence of the mutation can be verified by a number of standard techniques, for example by restriction analysis or by DNA sequencing.

One of ordinary skill in the art will appreciate that the DNA encoding the coat protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the coat protein. Examples of regulatory elements that can be incorporated into the vector include, but are not limited to, promoters, enhancers, terminators, and polyadenylation signals. One skilled in the art will appreciate that selection of suitable regulatory elements is dependent on the host cell chosen for expression of the genetically engineered coat protein and that such regulatory elements may be derived from a variety of sources, including bacterial, fungal, viral, mammalian or insect genes.

In certain embodiments, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids encoded by the heterologous nucleic acid sequence can be removed from the expressed coat protein prior to use according to methods known in the art. Alternatively, the amino acids corresponding to expression of heterologous nucleic acid sequences can be retained on the coat protein if they do not interfere with its subsequent assembly into VLPs.

In one embodiment of the present invention, the coat protein is expressed as a histidine tagged protein. The histidine tag can be located at the carboxyl terminus or the amino terminus of the coat protein. In certain embodiments, the coat protein comprises a histidine or similar tag at the C-terminus.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (e.g. E. coli, A. salmonicida or B. subtilis) or in a eukaryotic host (e.g. Saccharomyces or Pichia; mammalian cells, e.g. COS, NIH 3T3, CHO, BHK, 293 or HeLa cells; insect cells or plant cells). In certain embodiments, the coat protein is expressed in E. coli or P. pastoris.

If desired, the coat proteins can be purified from the host cells by standard techniques known in the art (see, for example, in Current Protocols in Protein Science, ed. Coligan, J. E., et al., Wiley & Sons, New York, N.Y.) and sequenced by standard peptide sequencing techniques using either the intact protein or proteolytic fragments thereof to confirm the identity of the protein.

ssRNA Template

The ssRNA template for use to prepare the ssRNA-VLPs may be, for example, synthetic ssRNA, a naturally occurring ssRNA, a modified naturally occurring ssRNA, a fragment of a naturally occurring or synthetic ssRNA, or the like.

Typically, the ssRNA for in vitro assembly is at least about 50 nucleotides in length and up to about 5000 nucleotides in length, for example, at least about 100, 250, 300, 350, 400, 450 or 500 nucleotides in length and up to about 5000, 4500, 4000 or 3500 nucleotides in length, or any amount therebetween. In certain embodiments, the ssRNA for in vitro assembly is between about 500 and about 3000 nucleotides in length, for example, between about 800 and about 3000 nucleotides in length, between about 1000 and about 3000 nucleotides in length, between about 1200 and about 3000 nucleotides in length, or between about 1200 and about 2800 nucleotides in length.

In certain embodiments, the ssRNA template is designed such that it does not include any ATG (AUG) start codons in order to minimize the chances of in vivo transcription of the sequences. The use of ssRNA templates including ATG start codons is not, however, excluded as in vivo transcription remains unlikely if the ssRNA is not capped.

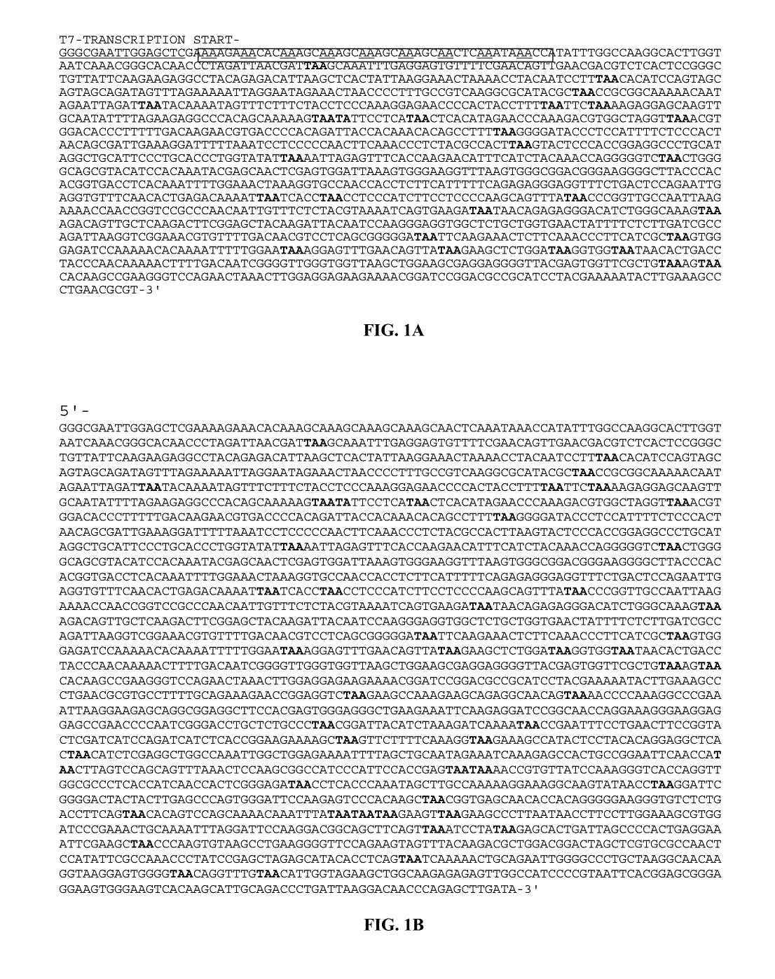

In certain embodiments, the ssRNA for in vitro assembly includes between about 38 and about 100 nucleotides from the 5′-end of the native PapMV RNA, which contain at least part of the putative packaging signal. ssRNA templates that do not include the putative packaging signal can also be used in certain embodiments. Non-limiting examples of sequences based on the PapMV genome that may be used to produce ssRNA templates are provided in FIG. 1 (SEQ ID NOs:1 and 6). Fragments of these sequences, as well as elongated versions of up to about 5000 nucleotides, are also contemplated for use to produce ssRNA templates in certain embodiments of the invention. In certain embodiments, the ssRNA for in vitro assembly comprises a RNA sequence corresponding to nucleotides 17 to 54 of SEQ ID NO:1. In certain embodiments, the ssRNA for in vitro assembly comprises a RNA sequence corresponding to nucleotides 17 to 63 of SEQ ID NO:1. In certain embodiments, the ssRNA for in vitro assembly comprises a RNA sequence corresponding to SEQ ID NO:1.

ssRNA sequences that are rich in A and C nucleotides are also known to assemble with PapMV coat protein (Sit, et al., 1994, Virology, 199:238-242). Accordingly, in certain embodiments, the ssRNA template is an A and/or C rich sequence, including poly-A and poly-C ssRNA templates. ssRNA templates based on all or part of the sequences of other potexviruses, such as potato virus X (PVX), clover yellow mosaic virus (CYMV), potato aucuba mosaic virus (PAMV) and malva mosaic virus (MaMV), are also contemplated for use in the process in some embodiments.

Preparation of ssRNA Template

The ssRNA template can be isolated and/or prepared by standard techniques known in the art (see, for example, Ausubel et al. (1994 & updates) Current Protocols in Molecular Biology, John Wiley & Sons, New York).

For example, for synthetic ssRNA, the sequence encoding the ssRNA template can be inserted into a suitable plasmid which can be used to transform an appropriate host cell. After culture of the transformed host cells under appropriate cell culture conditions, plasmid DNA can be purified from the cell culture by standard molecular biology techniques and linearized by restriction enzyme digestion.

The ssRNA is then transcribed using a suitable RNA polymerase and the transcribed product purified by standard protocols.

One skilled in the art will appreciate that the precise plasmid used is not critical to the invention provided that it is capable of achieving its desired purpose. Likewise the particular host cell used is not critical so long as it is capable of propagating the selected plasmid.

Shorter ssRNA templates may also be synthesized chemically using standard techniques. A number of commercial RNA synthesis services are also available.

The final ssRNA template may optionally be sterilized prior to use.

### In Vitro Assembly of VLPs

The assembly reaction is conducted in vitro using the prepared recombinant coat protein and ssRNA template. While both the recombinant coat protein and ssRNA template are typically purified prior to assembly, the use of crude preparations or partially purified coat protein and/or ssRNA template is also contemplated in some embodiments.

In general, preparations of recombinant coat proteins having identical amino acid sequences are employed in the assembly reaction, such that the final VLP when assembled comprises identical coat protein subunits. The use of preparations comprising a plurality of recombinant coat proteins having different amino acid sequences, such that the final VLP when assembled comprises variations in its coat protein subunits, are also contemplated in some embodiments.

The recombinant coat protein used in the assembly reaction is predominantly in the form of low molecular weight species consisting primarily of monomers and dimers, but also including other low molecular weight species of less than 20-mers. In the context of the present invention, a recombinant coat protein preparation is considered to be predominantly in the form of low molecular weight species when at least about 70% of the coat protein comprised by the preparation is present as low molecular weight species. In certain embodiments, at least about 75%, 80%, 85%, 90% or 95%, or any amount therebetween, of the coat protein in the recombinant coat protein preparation used in the assembly reaction is present as low molecular weight species. In certain embodiments of the present invention, the recombinant coat protein preparation is comprised of at least about 50% monomers and dimers, for example, about 60%, 70%, 75% or 80% monomers and dimers, or any amount therebetween.

The assembly reaction is conducted in a neutral aqueous solution and does not require any other particular ion. Typically, a buffer solution is used. The pH should be in the range of about pH6.0 to about pH9.0, for example, between about pH6.5 and about pH9.0, between about pH7.0 and about pH9.0, between about pH6.0 and about pH8.5, between about pH6.5 and about pH8.5, or between about pH7.0 and about pH8.5. The nature of the buffer is not critical to the invention provided that it can maintain the pH in the ranges described above. Examples of buffers for use within the pH ranges described above include, but are not limited to, Tris buffer, phosphate buffer, citrate buffer and the like.

The presence of high concentrations of sodium chloride (NaCl) may impact the assembly of PapMV coat protein. In certain embodiments, therefore, the assembly reaction is conducted in a solution that is substantially free of NaCl, for example, containing less than about 0.05M NaCl.

The assembly reaction can be conducted using various protein:ssRNA ratios. In general, a protein:ssRNA ratio between about 1:1 and about 50:1 by weight may be used, for example, between at least about 1:1, 2:1, 3:1, 4:1 or 5:1 by weight and no more than about 50:1, 40:1 or 30:1 by weight. In certain embodiments, the protein:ssRNA ratio used in the assembly reaction is between about 5:1 and about 40:1, or between about 10:1 and about 40:1 by weight, or any range therebetween.

The assembly reaction can be conducted at temperatures varying from about 2° C. to about 37° C., for example, between at least about 3° C., 4° C., 5° C., 6° C., 7° C., 8° C., 9° C. or 10° C. and about 37° C., 35° C., 30° C. or 28° C. In certain embodiments, the assembly reaction is conducted at a temperature between about 15° C. and about 37° C., for example, between about 20° C. and about 37° C., or between about 22° C. and about 37° C., or any range therebetween.

The assembly reaction is allowed to proceed for a sufficient period of time to allow VLPs to form. The time period will vary depending on the concentrations of recombinant coat protein and ssRNA employed, as well as the temperature of the reaction, and can be readily determined by the skilled worker. Typically time periods of at least about 60 minutes are employed. Assembly of the coat protein into VLPs can be monitored if required by standard techniques, such as dynamic light scattering or electron microscopy.

After the assembly reaction has been allowed to proceed for an appropriate length of time, the VLPs may be subjected to a “blunting” step to remove RNA protruding from the particles. The blunting reaction is achieved using a nuclease capable of cutting RNA. Various nucleases are commercially available and conditions for their use are known in the art.

The VLPs once assembled can be purified from other reaction components by standard techniques, such as dialysis, diafiltration or chromatography.

The ssRNA-VLP preparation can optionally be concentrated (or enriched) by, for example, ultracentrifugation or diafiltration, either before or after the purification step(s). ssRNA-VLPs can be visualized using standard techniques, such as electron microscopy, if desired.

### Pharmaceutical Compositions

In certain embodiments, the present invention provides for pharmaceutical compositions comprising an effective amount of the PapMV or PapMV ssRNA-VLPs and one or more pharmaceutically acceptable carriers, diluents and/or excipients. If desired, other active ingredients may be included in the compositions, for example, additional immunotherapeutics, chemotherapeutics, therapeutic cancer vaccines or the like. Some embodiments of the invention relate to therapeutic combinations that comprise the PapMV or PapMV ssRNA-VLPs and another cancer therapeutic, such as an immunotherapeutic or a chemotherapeutic as described herein, in which the PapMV or PapMV ssRNA-VLPs and the other cancer therapeutic are formulated as separate compositions, but are for use in combination.

The pharmaceutical compositions can be formulated for administration by a variety of routes. For example, the compositions can be formulated for oral, topical, rectal, nasal or parenteral administration or for administration by inhalation or spray. The term parenteral as used herein includes subcutaneous, intravenous, intramuscular, intrathecal, intrasternal injection or infusion techniques. Intranasal administration to the subject includes administering the composition to the mucous membranes of the nasal passage or nasal cavity of the subject. Intra-tumoral administration is also contemplated in some embodiments.

Compositions formulated as aqueous suspensions may contain the PapMV or PapMV ssRNA-VLPs in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

In certain embodiments, the pharmaceutical compositions may be formulated as a dispersible powder or granules, which can subsequently be used to prepare an aqueous suspension by the addition of water. Such dispersible powders or granules provide the PapMV or PapMV ssRNA-VLPs in admixture with one or more dispersing or wetting agents, suspending agents and/or preservatives. Suitable dispersing or wetting agents and suspending agents are exemplified by those already mentioned above. Additional excipients, for example, colouring agents, can also be included in these compositions.

Pharmaceutical compositions may also be formulated as oil-in-water emulsions in some embodiments. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

In certain embodiments, the pharmaceutical compositions may be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the pharmaceutical compositions may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Sterile compositions can be prepared for example by incorporating the PapMV or PapMV ssRNA-VLPs in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some exemplary methods of preparation are vacuum-drying and freeze-drying techniques which yield a powder of the active ingredient plus any additional desired ingredient from a previously sterile-filtered solution thereof.

Contemplated for use in certain embodiments of the invention are various mechanical devices designed for pulmonary or intranasal delivery of therapeutic products, including but not limited to, nebulizers, metered dose inhalers, powder inhalers and nasal spray devices, all of which are familiar to those skilled in the art.

All such devices require the use of formulations suitable for the dispensing of the PapMV or PapMV ssRNA-VLPs. Typically, each formulation is specific to the type of device employed and may involve the use of an appropriate propellant material, in addition to the usual diluents, adjuvants and/or carriers useful in therapy as would be understood by a worker skilled in the art. Also, the use of liposomes, microcapsules or microspheres, inclusion complexes, or other types of carriers is contemplated in certain embodiments.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

Also encompassed in some embodiments of the present invention are pharmaceutical compositions comprising PapMV or PapMV ssRNA-VLPs in combination with one or more commercially available chemotherapeutics or immunotherapeutics.

### Uses

The present invention relates generally to methods and uses of PapMV and PapMV ssRNA-VLPs in the treatment of cancer, either alone or in combination with one or more other cancer therapies. In this context, treatment of cancer may result in, for example, one or more of a reduction in the size of a tumour, the slowing or prevention of an increase in the size of a tumour, an increase in the disease-free survival time between the disappearance or removal of a tumour and its reappearance, prevention of an initial or subsequent occurrence of a tumour (e.g. metastasis), an increase in the time to progression, reduction of one or more adverse symptoms associated with a tumour, or an increase in the overall survival time of a subject having cancer.

Without being limited to any particular theory or mechanism, it is believed that administration of the PapMV ssRNA-VLPs to patients with cancer increases the pool of immune cells that are involved in fighting the cancer. While cancer patients are known to mount an anti-cancer immune response, this response is usually insufficient to impact cancer growth or progression. Administration of the PapMV ssRNA-VLPs in order to increase the existing pool of immune cells and/or to stimulate an anti-cancer immune response should, therefore, increase the efficacy of this response against the cancer. For similar reasons, the PapMV ssRNA-VLPs should also have efficacy in improving the effects of known anti-cancer therapies. When a selected anti-cancer therapy (for example, a chemotherapeutic) is toxic to, or otherwise results in a decrease in, immune cells, decreased doses of this drug may need to be used in combination with the PapMV ssRNA-VLPs to avoid the possibility of the chemotherapeutic counteracting the immunomodulatory effects of the PapMV ssRNA-VLPs.

In combination therapies, it is contemplated that in most embodiments, the PapMV or PapMV ssRNA-VLPs will enhance the effects of the other therapy or therapies in the combination. In various embodiments depending on the particular combination, the effect of the PapMV or PapMV ssRNA-VLPs with the other therapy/therapies may be additive, more than additive or synergistic.

Examples of cancers which may be may be treated or stabilized in accordance with certain embodiments of the invention include, but are not limited to, haematologic neoplasms (including leukaemias, myelomas and lymphomas); carcinomas (including adenocarcinomas and squamous cell carcinomas); melanomas and sarcomas. Carcinomas and sarcomas are also frequently referred to as “solid tumours.” Examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, in certain contexts may also be considered to be solid tumours.

In certain embodiments, the PapMV or PapMV ssRNA-VLPs may be used in treatment of a solid tumour. In certain embodiments, the PapMV or PapMV ssRNA-VLPs may be used in treatment of a cancer for which immunotherapy is known to be particularly effective, for example, bladder cancer, breast cancer, colon cancer, kidney cancer, lung cancer, prostate cancer, leukemia, lymphoma, multiple myeloma and melanoma. In certain embodiments, the invention relates to the use of the PapMV or PapMV ssRNA-VLPs in the treatment of a cancer other than lung cancer.

The cancer to be treated may be indolent or it may be aggressive. In various embodiments, the invention contemplates the use of PapMV or PapMV ssRNA-VLPs in the treatment of refractory cancers, advanced cancers, recurrent cancers or metastatic cancers. One skilled in the art will appreciate that many of these categories may overlap, for example, aggressive cancers are typically also metastatic.

Various modes of administration of the PapMV or PapMV ssRNA-VLPs are contemplated depending on the cancer to be treated, including systemic administration and local administration. An appropriate route of administration can be readily determined by the skilled person having regard to the cancer to be treated. Some embodiments comprise the systemic administration of PapMV or PapMV ssRNA-VLPs in the treatment of cancer, for example, subcutaneous, intravenous, intramuscular or intranasal administration. In certain embodiments, the invention relates to local administration of PapMV or PapMV ssRNA-VLPs in the treatment of cancer, for example, intratumoral or peri-tumoral administration. In certain embodiments, the invention relates to local administration of PapMV or PapMV ssRNA-VLPs by a route other than a pulmonary route.

In certain embodiments, the invention relates to methods of using PapMV or PapMV ssRNA-VLPs as a single agent to treat cancer. Some embodiments relate to the use of the PapMV or PapMV ssRNA-VLPs alone to inhibit tumour growth. Some embodiments relate to methods of inhibiting tumour growth that comprise intra-tumoral administration of the PapMV or PapMV ssRNA-VLPs.

In some embodiments, the invention relates to methods of using PapMV or PapMV ssRNA-VLPs in combination with one or more other cancer therapies to treat cancer. Some embodiments relate to the use of the PapMV or PapMV ssRNA-VLPs VLPs in combination with one or more other cancer therapies to inhibit tumour growth and/or metastasis. Other cancer therapies may include, for example, immunotherapeutics, chemotherapeutics, radiotherapy and virotherapy.

When used as part of a combination therapy, various orders of administration of the PapMV or PapMV ssRNA-VLPs and the other cancer therapy or therapies are contemplated. Certain embodiments of the invention relate to administration of the PapMV or PapMV ssRNA-VLPs prior to or concomitantly with the administration of the other therapy or therapies. Concomitant administration in this context includes both simultaneous administration of the PapMV or PapMV ssRNA-VLPs and the other therapy, as well as administration of the PapMV or PapMV ssRNA-VLPs within a short time period (before or after) administration of the other therapy or therapies, for example, within two hours or less, 90 minutes or less, 60 minutes or less, or 30 minutes or less, of administration of the other therapy or therapies.

Some embodiments relate to the administration of the PapMV or PapMV ssRNA-VLPs subsequent to the other therapy or therapies.

Regardless of the order of administration, further administration of the one or more “boosters” of the PapMV or PapMV ssRNA-VLPs subsequent to the administration of the other therapy or therapies is also contemplated in some embodiments.

Some embodiments of the invention relate to administration of the PapMV or PapMV ssRNA-VLPs prior to administration of the one or more other anti-cancer therapies. Administration of the PapMV or PapMV ssRNA-VLPs prior to another therapy may, for example, “prime” the immune system so that the effect of the subsequently administered therapeutic is enhanced. In this context, administration of the PapMV or PapMV ssRNA-VLPs and therapeutic agent(s) are separated by a defined time period that may be short (for example in the order of minutes) or more extended (for example in the order of hours, days or weeks).

Typically, when the PapMV or PapMV ssRNA-VLPs are administered prior to or subsequent to another therapy, the time period between administration of the PapMV or PapMV ssRNA-VLPs and the other therapy will be at least 30 minutes, for example, at least 60 minutes, at least 90 minutes or 120 minutes. In some embodiments, the time period between administration of the PapMV or PapMV ssRNA-VLPs and the other therapy may be at least 3 hours, at least 4 hours, at least 5 hours or at least 6 hours. In some embodiments, the time period between administration of the PapMV or PapMV ssRNA-VLPs and the other therapy may be between about 2 hours and about 48 hours, for example, between about 2 hours and about 36 hours, between about 2 hours and about 24 hours, or between about 2 hours and about 18 hours. In some embodiments, In some embodiments, the time period between administration of the PapMV or PapMV ssRNA-VLPs and the other therapy may be between about 3 hours and about 24 hours, between about 4 hours and about 24 hours or between about 5 hours and about 24 hours.

Some embodiments relate to the use of PapMV or PapMV ssRNA-VLPs as an adjunct therapy, for example, as an adjunct therapy to radiotherapy or to surgery. In this context, it is contemplated that stimulation of the innate immune system by the PapMV or PapMV ssRNA-VLPs may help to eliminate any tumour cells remaining after radiotherapy or surgery, or it may weaken the tumour cells prior to radiotherapy. Such adjunct therapy may help to increase the success rate of radiotherapy and surgical interventions.

In certain embodiments, the invention relates to methods of using PapMV or PapMV ssRNA-VLPs in combination with one or more cancer immunotherapeutics to inhibit tumour growth. Some embodiments of the invention relate to methods of using PapMV or PapMV ssRNA-VLPs in combination with one or more cancer immunotherapeutics to inhibit tumour metastasis. Various cancer immunotherapeutics are known in the art and include, for example, monoclonal antibodies (such as alemtuzumab (Campath®), cetuximab (Erbitux®), panitumumab (Vectibix™) rituximab (e.g. Rituxan®), trastuzumab (Herceptin®) and ipilimumab (Yervoy™)), cancer vaccines (such as sipuleucel-T (Provenge®) and other dendritic cell vaccines, tumour cell vaccines, PBMC vaccines and viral vector-based vaccines) and non-specific immunotherapeutics (such as Interleukin-2 (e.g. Proleukin®), interferon (IFN)-alpha and other cytokines; thalidomide, imiquomod and lenalidomide). Use of the PapMV or PapMV ssRNA-VLPs in combination with other immunotherapies, such as adoptive cell therapy (ACT), are also contemplated in some embodiments. In certain embodiments, the invention relates to methods of using PapMV or PapMV ssRNA-VLPs in combination with one or more cell-based cancer immunotherapeutics such as dendritic cells, PBMCs, tumour cells, and the like. In certain embodiments, the PapMV or PapMV ssRNA-VLPs may be administered in combination with a dendritic cell-based cancer therapy.

As is known in the art, dendritic cell-based cancer therapy is typically based on dendritic cells derived from in vitro expansion of monocyte-derived progenitors obtained from a patient and subsequently loaded with one or more tumour-associated antigens. The antigens can be incubated with the dendritic cells in various forms, including for example peptides, recombinant proteins, plasmid DNA, formulated RNA, or recombinant viruses. Cancer vaccines based on naïve dendritic cells are also being developed for intratumoral administration and use in combination with other therapeutic modalities, such as radiotherapy.

Certain embodiments of the invention relate to the use of PapMV or PapMV ssRNA-VLPs to improve a cancer immunotherapy in a subject by administering to the subject an effective amount of the PapMV or PapMV ssRNA-VLPs prior to, concomitantly with, or subsequent to administration of the cancer immunotherapy. In some embodiments, the PapMV or PapMV ssRNA-VLPs is used to improve a cancer immunotherapy comprising dendritic cells loaded with a cancer specific antigen. In some embodiments, the PapMV or PapMV ssRNA-VLPs are administered to the patient as a pretreatment in order to improve the efficacy of the dendritic cell treatment through stimulation of the innate immunity of the patient prior to administration of the antigen-loaded dendritic cells. Concomitant and subsequent administration of the PapMV or PapMV ssRNA-VLPs are also contemplated in alternative embodiments.

Certain embodiments relate to methods of using PapMV or PapMV ssRNA-VLPs in combination with one or more cancer chemotherapeutics to inhibit growth and/or metastasis of a cancer. Various chemotherapeutics are known in the art and include those that are specific for the treatment of a particular type of cancer as well as broad spectrum chemotherapeutics that are applicable to a range of cancers. Examples of chemotherapeutics include, but are not limited to, amifostine (e.g. Ethyol®), L-asparaginase, capecitabine (e.g. Xeloda®), carboplatin, cisplatin, cyclophosphamide, cytarabine, dacarbazine, docetaxel (e.g. Taxotere®), doxazosin (e.g. Cardura®), doxorubicin (e.g. Adriamycin®), edatrexate (10-ethyl-10-deaza-aminopterin), epi-doxorubicin (epirubicin), estramustine, etoposide, finasteride (e.g. Proscar®), fluorodeoxyuridine (FUdR), 5-fluorouracil (5-FU), flutamide (e.g. Eulexin®), gemcitabine (e.g. Gemzar®), goserelin acetate (e.g. Zoladex®), idarubicin, ifosfamide, irinotecan (CPT-11, e.g. Camptosar®), levamisole, leucovorin, liarozole, loperamide (e.g. Imodium®), melphalan, methotrexate, methyl-chloroethyl-cyclohexyl-nitrosourea, mitoxantrone (e.g. Novantrone®), nilutamide (e.g. Nilandron®), nitrosoureas, oxaliplatin, paclitaxel (e.g. Taxol®), pegaspargase (e.g. Oncaspar®), platinum analogues, prednisone (e.g. Deltasone®), procarbazine (e.g. Matulane®), porfimer sodium (e.g. Photofrin®), tamoxifen, temozolomide, terazosin (e.g. Hytrin®), topotecan (e.g. Hycamtin®), tretinoin (e.g. Vesanoid®), vincristine and vinorelbine tartrate (e.g. Navelbine®).

In certain embodiments, the PapMV or PapMV ssRNA-VLPs may be administered in combination with a chemotherapeutic that also has immunomodulatory effects. In some embodiments, the PapMV or PapMV ssRNA-VLPs may be administered in combination with a chemotherapeutic that also has immunomodulatory effects, wherein the dose of chemotherapeutic that is administered is reduced compared to the dose that would normally be administered in the absence of the PapMV or PapMV ssRNA-VLPs. For example, cyclophosphamide is known to exhibit immunomodulatory effects that are dependent on the dosage administered (Motoyoshi, et al., 2006, Oncology Reports, 16:141-146). In certain embodiments, therefore, the use of PapMV or PapMV ssRNA-VLPs in combination with low-dose cyclophosphamide to treat cancer is contemplated. The use of PapMV or PapMV ssRNA-VLPs in combination with low doses of other chemotherapeutics is also contemplated in some embodiments.

Certain embodiments of the invention relate to the methods and uses of the PapMV or PapMV ssRNA-VLPs in combination with radiotherapy for the treatment of cancer. In this context, it is contemplated that stimulation of the innate immune system by the PapMV or PapMV ssRNA-VLPs may enhance the effects of radiotherapy and/or help to eliminate any tumour cells remaining after therapy.

Certain embodiments of the invention contemplate the use of the PapMV or PapMV ssRNA-VLPs to enhance known combination therapies, for example, combinations of chemotherapeutics, combinations of chemotherapeutic(s) and immunotherapeutic(s), combination of radiotherapy with chemotherapeutic(s) or combination of radiotherapy with immunotherapeutic(s). Such combinations are well known in the art for treatment of specific cancers at various stages.

Some embodiments of the invention relate to methods and uses of the PapMV or PapMV ssRNA-VLPs in combination with radiotherapy and another cancer therapeutic, such as a chemotherapeutic or an immunotherapeutic. For example, combination of radiotherapy and low dose cyclophosphamide has been found to be useful in the treatment of certain cancers, including lymphoma, and could be further combined with PapMV or PapMV ssRNA-VLPs to enhance the effects of this combination therapy.

In certain embodiments, the use of the PapMV or PapMV ssRNA-VLPs in combination with virotherapy is contemplated. Oncolytic virotherapy is currently being developed as a targeted approach for the treatment of cancer. Several oncolytic virus-based therapies are undergoing clinical trials and include therapies based on herpes simplex virus (HSV), reovirus, vaccinia virus (VV), adenovirus, measles virus and vesicular stomatatis virus (VSV). Combination of PapMV or PapMV ssRNA-VLPs with virotherapy approaches is contemplated as a means to improve the efficacy of the virotherapy in reducing tumour growth and/or metastasis.

The amount of PapMV or PapMV ssRNA-VLPs to be administered can be estimated initially, for example, in animal models, usually in rodents, rabbits, dogs, pigs or primates. The animal model may also be used to determine the appropriate concentration range and route of administration. Such information can then be used to determine useful doses and routes for administration in the patient to be treated. Exemplary doses of the PapMV or PapMV ssRNA-VLPs include doses between about 10 μg and about 10 mg of protein, for example, between about 10 μg and about 5 mg of protein, between about 40 μg and about 5 mg of protein, between about 80 μg and about 5 mg of protein, between about 40 μg and about 2 mg of protein, or between about between about 80 μg and about 2 mg of protein.

### Pharmaceutical Packs & Kits

Certain embodiments of the invention provide for pharmaceutical packs and kits comprising PapMV or PapMV ssRNA-VLPs for use in cancer therapy. When the PapMV or PapMV ssRNA-VLPs are for use in combination with another cancer therapeutic, for example a chemotherapeutic or immunotherapeutic, the pharmaceutical pack or kit may contain a therapeutic combination for use in the treatment of cancer that comprises the PapMV or PapMV ssRNA-VLPs and the cancer therapeutic.

Individual components of the pack or kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the PapMV or PapMV ssRNA-VLPs and, when present, the other cancer therapeutic.

One or more of the components of the pack or kit may optionally be provided in dried or lyophilised form and the kit can additionally contain a suitable solvent for reconstitution of the lyophilised component(s).

In those embodiments in which one or more components are provided as a solution, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

Irrespective of the number or type of containers, the kits of the invention also may comprise an instrument for assisting with the administration of the PapMV or PapMV ssRNA-VLPs to a patient. Such an instrument may be an inhalant, nasal spray device, nebulizer, syringe, pipette, eye dropper or similar medically approved delivery vehicle.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### PapMV ssRNA-VLPs Inhibit Tumour Growth and Potentiate the Anti-Cancer Effects of Dendritic Cells In Vivo

PapMV VLPs comprising ssRNA (PapMV ssRNA-VLPs) used in this Example were prepared as described in International Patent Application Publication No. WO2012/155262 (see also Example 2). The coat protein of the VLPs was the modified PapMV coat protein, PapMV CPsm (SEQ ID NO:5; see FIG. 3).

### Summary

Vaccination is a promising cancer therapy, especially when this involves dendritic cells, which are responsible for the presentation of antigens to lymphocytes (Banchereau and Palucka 2005, Nat Rev Immunol, 5(4):296-306). This method, however, is not fully effective for treatment of tumours. Addition of a TLR7 ligand leading to the production of IFN-α could help improve the anti-tumour response generated by this type of vaccine. PapMV ssRNA-VLPs, which are ligands of TLR7 and induce the production of IFN-α, could serve as an immunomodulator. PapMV ssRNA-VLPs are known to be taken up by dendritic cells and to induce a cytotoxic cellular immune response. The objectives of this study were to characterize the effect of PapMV ssRNA-VLPs on the anti-tumour response against murine melanoma B16-OVA in a subcutaneous model, as well as a model of lung metastases. As a result of this study, it was determined that intratumoral immunization with PapMV ssRNA-VLPs increased the delay in tumour growth resulting from immunization with dendritic cells and, in addition, when injected before dendritic cells loaded with OVA, PapMV ssRNA-VLPs increased the reduction in pulmonary metastases. PapMV ssRNA-VLPs therefore have promising capabilities to act as an immunomodulator in the anti-tumour response.

### Introduction

Vaccines currently under development for cancer therapy are based on the activation of antigen presenting cells, the generation of an inflammatory environment as well as an increase the immunogenicity of tumour cells Immunization with dendritic cells results in some, but not completely effective, tumour regression. A TLR7 ligand, which induces the production of IFN-α, could serve to enhance the effect of dendritic cells. IFN-α is involved in the maturation of dendritic cells, as well as activation of the cytotoxic anti-tumoural response (Diamond, et al., 2011, J Exp Med, 208(10): 1989-2003).

PapMV ssRNA-VLPs are phagocytosed by dendritic cells, where the ssRNA is recognized by TLR7, leading to production of IFN-a. This then induces protective cytotoxic cell-mediated immunity.

### Materials and Methods

B16 mouse melanoma cell line expressing OVA (B16-OVA) and another variant of this cell line expressing luciferase (B16-OVA-ofl) were used for the experiments.

Bone marrow-derived dendritic cells (BMDC) were generated from bone marrow of male mice following incubation in the presence of GM-CSF and IL-4 for 6 days. The BMDC were then stimulated with LPS and loaded with the OVA peptide.

Plasmid SRa containing the oFL gene was transfected into B16-OVA tumour cells by the calcium phosphate method. B16-OVA-ofL tumours can be detected by luminescence before they can be detected visually, allowing subcutaneous growth of the tumour to be followed.

The kinetics of tumour growth in vivo were measured by caliper and by intraperitoneal (i.p.) injection of 20 ug luciferin, followed by analysis using the In Vivo Imaging System (IVIS; PerkinElmer, Waltham, Mass.).

Analysis of the immune response in the lungs was carried out by infusing with PBS—2 mM EDTA and then analysis by FACS. Complement depletion in the mouse was achieved by i.p. administration of 20 μg cobra venom factor.

Flow cytometry was carried out using the BD LSRFortessa™ (BD Biosciences, San Jose, Calif.) and data were analyzed using the FlowJo software (FlowJo, LLC, Ashland, Oreg.).

### Subcutaneous Tumour Model

For the local tumour growth model, 1×10⁵ or 5×10⁵ B16-OVA cells were injected subcutaneously (s.c.). Around 7 days post injection, the tumour began to be visible. Treatments were administered at day 7 and/or day 12 post tumour cell inoculation Immune response was analyzed at day 16 post-inoculation by flow cytometry.

The treatments tested were:
100 μg PapMV ssRNA-VLPs intravenous (i.v.)
100 μg PapMV ssRNA-VLPs intratumoral (i.t.)
100 μg PapMV ssRNA-VLPs i.t. with 1.25×10⁶ bone marrow derived dendritic cells loaded with OVA (BMDC-OVA) s.c. in the opposite flank 6 h later.

These treatments were compared to the controls:
100 μl PBS i.v.
100 μl PBS i.t.
100 μl PBS+1.25×10⁶ BMDC-OVA 6 h later.

### Metastasis Model

For the induction of metastasis in the lung, 5×10⁵ B16-OVA or B16-OVA-ofl were injected i.v. Treatments were administered at 7 days post tumour cells inoculation. Immune response was analyzed at day 14 post-inoculation by flow cytometry.

Treatments tested were:
100 μg PapMV ssRNA-VLPs i.v.
100 μg PapMV ssRNA-VLPs i.v. with 1.25×10⁶ BMDC-OVA i.v. 6 h later

These treatments were compared to the controls:
100 μl PBS i.v.
100 μl PBS i.v.+1.25×10⁶ BMDC-OVA i.v. 6 h later.

### Results and Discussion

Effect of PapMV ssRNA-VLPs in the Anti-Tumour Response Against Subcutaneous Melanoma

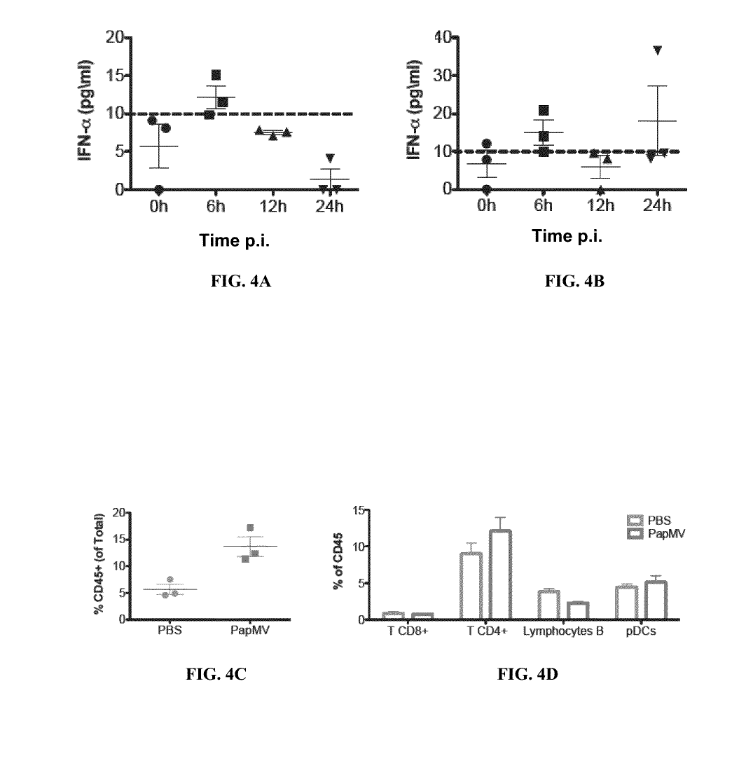

The results are shown in FIGS. 4-8.

Intratumoral (i.t.) immunization with PapMV ssRNA-VLPs induced production of IFN-α 6 h post immunization (p.i.) (FIG. 4A,B). Luminex verified the production of additional cytokines.

When PapMV ssRNA-VLPs were injected i.t. at day 12 post tumour cells inoculation, an increase in immune cell (CD45⁺) infiltration into the tumour was observed 24 h later (FIG. 4C,D). The proportion of different types of immune cells did not seem to be changed with the treatment.

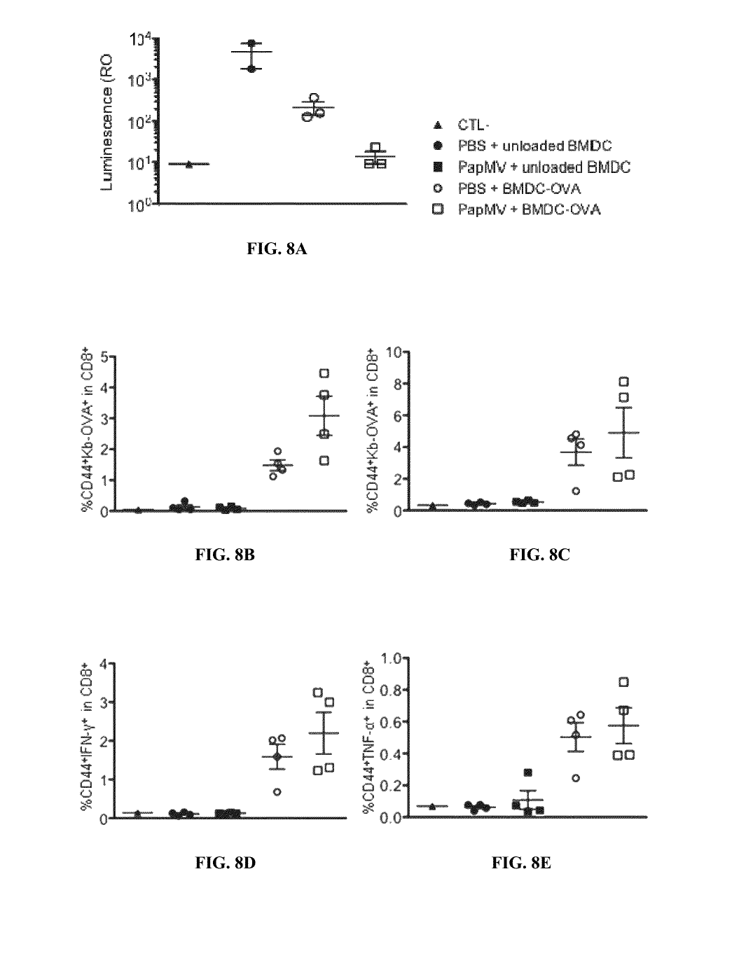

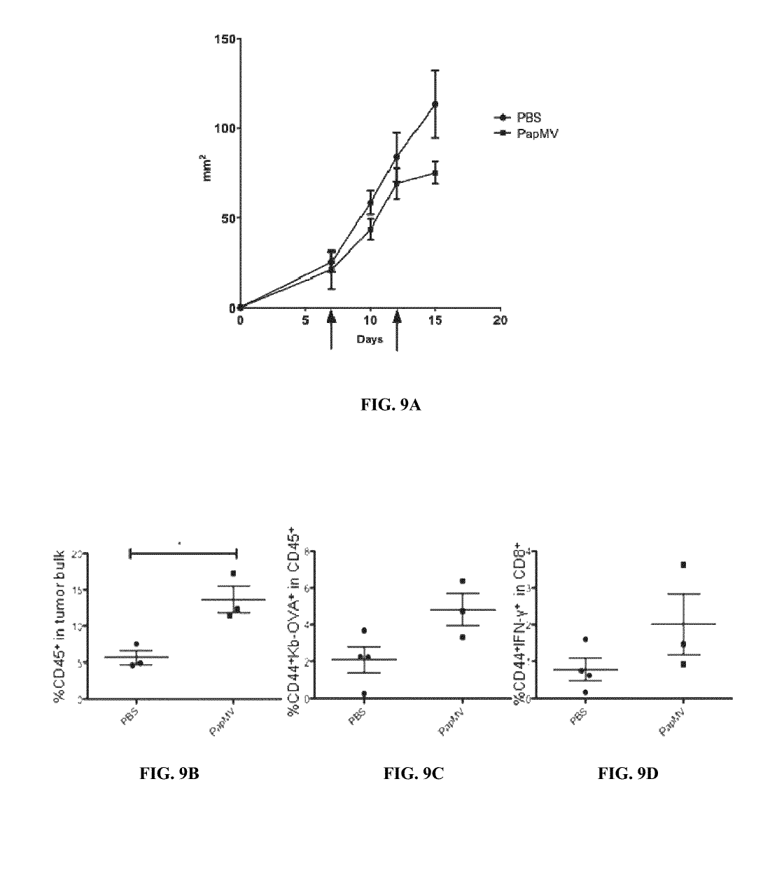

Intravenous injection of PapMV ssRNA-VLPs at day 7 and 12 did not have a significant effect on the tumour growth kinetics. However, i.t. injection of PapMV ssRNA-VLPs decreased the growth rate of B16-OVA and increased the proportion of OVA-specific CD8⁺ T cells (FIG. 8).

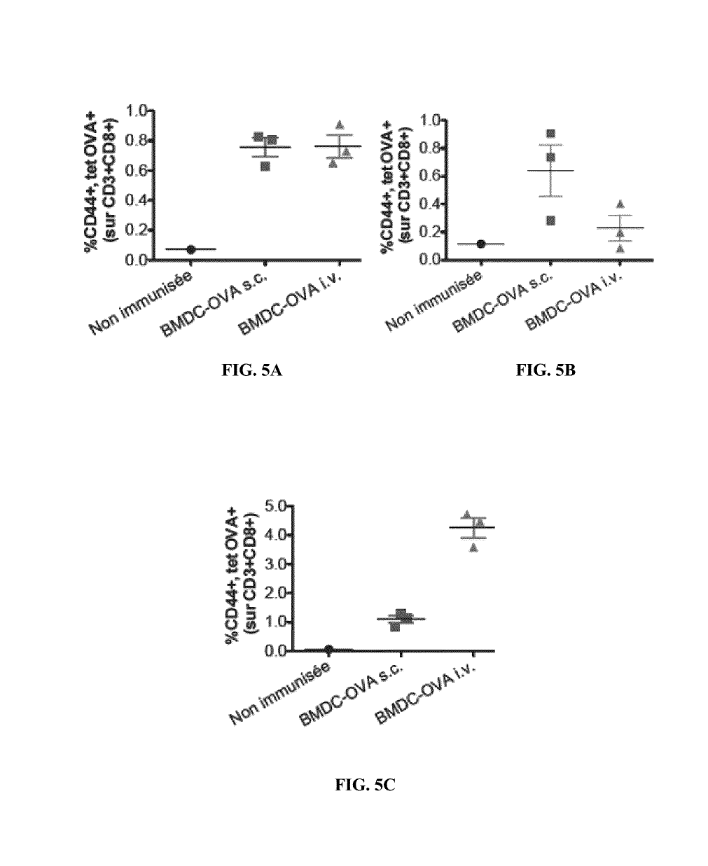

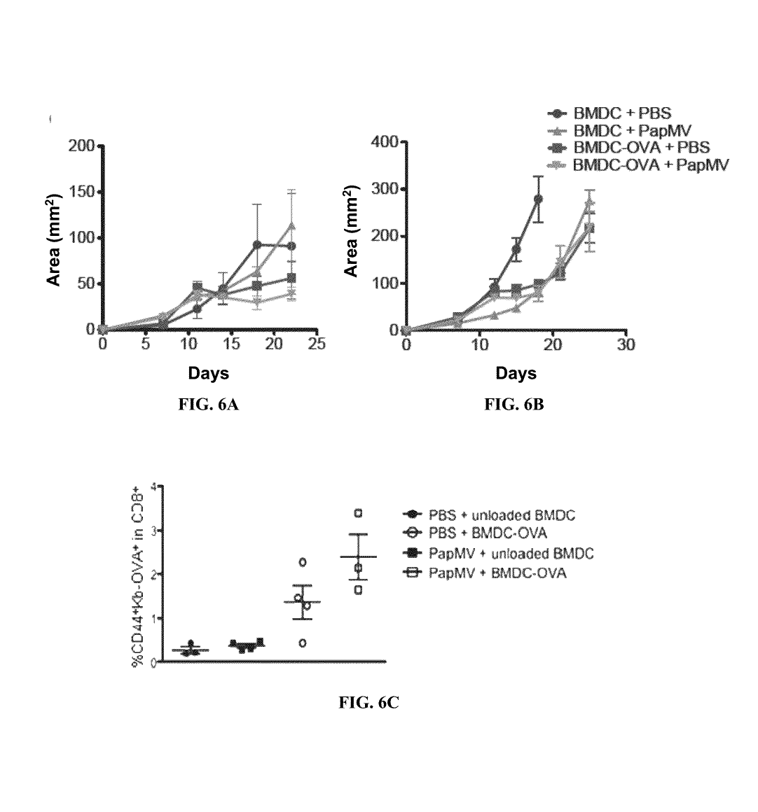

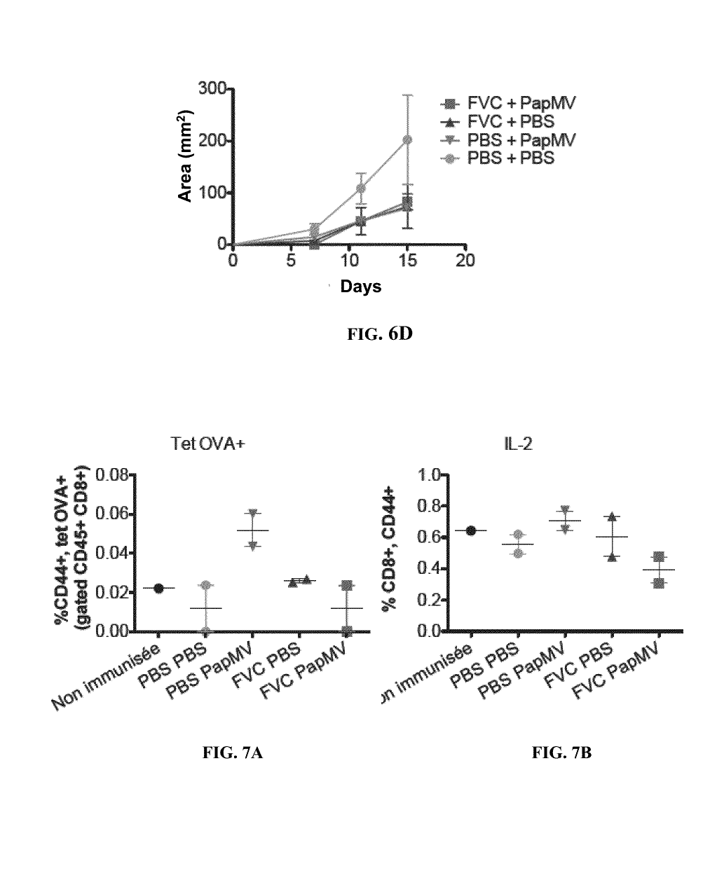

Subcutaneous (s.c.) immunization with dendritic cells loaded with OVA generated CD8⁺ OVA-specific T lymphocytes, resulting in an inhibition of tumour growth (FIG. 5). PapMV ssRNA-VLPs also increased the therapeutic effect of BMDC-OVA treatment (slowed the growth kinetics and increased the proportion of OVA-specific CD8⁺ T cells) (FIG. 6A-C). Finally, complement depletion in these situations did not increase the beneficial effect of the PapMV treatment (FIG. 6D).

### Effect of PapMV in the Anti-Tumour Response Against Pulmonary Metastases

The results are shown in FIGS. 7 and 8.

Intravenous injection of PapMV ssRNA-VLPs at day 7 did not induce production of OVA-specific CD8⁺ T cells in the lungs, the lymph nodes or the spleen (FIG. 7). Complement depletion did not change this result. However, injection of PapMV ssRNA-VLPs i.v. 6 h before BMDC-OVA immunization increased the proportion of OVA-specific CD8⁺ T cells in the lung and the spleen and reduced the luminescence production by the lung homogenate following addition of luciferin, thus suggesting a reduction in the number of live tumour cells (FIG. 8).

### Conclusion

This study demonstrates that PapMV ssRNA-VLPs alone have an effect on tumour growth of skin melanoma tumours. When combined with immunization with dendritic cells loaded with OVA, PapMV ssRNA-VLPs improve the anti-tumour response over dendritic cells alone. Although in the model of pulmonary metastases, PapMV ssRNA-VLPs alone showed no effect, the anti-tumour effect of immunization with dendritic cells loaded with OVA was substantially improved by administration of PapMV ssRNA-VLPs. Intranasal immunization of the PapMV ssRNA-VLPs may help to further improve these results by promoting the distribution of the PapMV ssRNA-VLPs to the lungs.

### Example 2

### Exemplary Process for Preparing PapMV ssRNA-VLPs

Production of Recombinant Coat Protein (rCP)

In brief, the PapMV CP harbouring a 6× His-tag (SEQ ID NO:5; see FIG. 3(B)) was cloned into the pQE80 vector (QIAGEN) flanked by the restriction enzyme NcoI and BamHI and under the control of the T5 promoter. E. coli BD-792 were transformed with the plasmid and grown in standard culture medium. Protein expression was induced by addition of IPTG (0.7-1 mM IPTG for 6-9 h at 22-25° C.) to the culture medium. At the end of the induction period, cells were harvested, suspended in lysis buffer (10 mM Tris pH 8.0, 500 mM NaCl) and ruptured mechanically using a French press, homogenizer or sonicator. Cell lysate was clarified by removal of genomic DNA by standard DNase treatment and removal of large cell fragments and membranes by centrifugation or tangential flow filtration (300 kDa to 0.45 μm MWCO membranes). rCP was captured on an ion-matrix affinity resin and eluted with imidazole using standard procedures. The PapMV coat protein can be eluted with between 250 mM and 1M imidazole. Elution could also be achieved using a pH gradient. The rCP was subsequently purified from endotoxins by anion exchange chromatography/filtration and from small low MW molecules by tangential flow filtration (0 to 30 kDa MWCO membranes). Any contaminating imidazole present in the rCP solution was removed by dialysis or tangential flow filtration (5 to 30 kDa MWCO membranes). The final rCP protein solution was sterilized by filtration.

Production of ssRNA Template (SRT)

The sequence of the DNA encoding the SRT is provided in FIG. 1 [SEQ ID NO:1]. The SRT is based on the genome of PapMV and harbours the PapMV coat protein nucleation signal at the 5′-end (boxed in FIG. 1). The remaining nucleotide sequence is poly-mutated in that all ATG codons have been mutated for TAA stop codons. The first 16 nucleotides of the sequence (underlined in FIG. 1) comprise the T7 transcription start site located within the pBluescript expression vector and are present within the RNA transcript. Pentameric repeats are underlined in FIG. 1. The entire transcript is 1522 nucleotides in length.

DNA corresponding to the SRT was inserted into a DNA plasmid including a prokaryotic RNA polymerase promoter using standard procedures. The recombinant plasmid was used to transform E. coli cells and the transformed bacteria were subsequently grown in standard culture medium. The plasmid DNA was recovered and purified from the cell culture by standard techniques, then linearized by cleavage with the restriction enzyme MluI at the point in the DNA sequence immediately after the last nucleotide of the SRT sequence.

Transcription of SRT was conducted with T7 RNA polymerase using the RiboMAX™ kit (Promega, USA) following the manufacturer's recommended protocol. The expression vector was designed such that transcripts originating from the RNA polymerase promoter were released from the DNA template at the DNA point of cleavage. The SRT was purified to remove DNA, protein and free nucleotides by tangential flow filtration using a 100 kDa MWCO membrane. The final RNA solution was sterilized by filtration.

Production of rVLPs

rVLPs were assembled in vitro by combining the rCP and SRT. The assembly reaction was conducted in a neutral buffered solution (10 mM Tris-HCl pH 8) and using a protein:RNA ratio between 15-30 mg of protein for 1 mg RNA. The newly assembled rVLPs were incubated with a low amount of RNase (0.0001 μg RNAse per μg RNA) to remove any RNA protruding from the rVLPs. The blunted-rVLPs were then purified from contaminants and free rCP (unassembled monomeric rCP) by diafiltration using 10-100 kDa MWCO membranes. The final rVLP liquid suspension was sterilized by filtration.

### Example 3

### Administration of PapMV ssRNA-VLPs via Various Routes Stimulates the Innate Immune System

### Intranasal Administration

Mice (5 per group) were treated intranasally either once or twice (at a 7 day interval) with 60 μg PapMV ssRNA-VLPs or with the control buffer (10 mM Tris HCl pH8). Broncho-alveolar lavage (BAL) was performed 6 hours after treatment and screened for the presence of cytokines using Luminex technology (Milliplex Mouse cytokine premixed 32-plex immunoassay kit; Millipore).

The results are shown in FIG. 10(A)-(R). Both treatment regimens induced cytokine and chemokine production, with 2 treatments being more effective than one.

### Intravenous Administration

Two groups of C57BL/6 mice, as well as TLR-7 knockout (KO) and MYD88 KO mice (4 mice per group) were immunized i.v. with 100 μg PapMV ssRNA-VLP or 100 μl PBS. One group of C57BL/6 mice had first been treated by injection i.p. with 500 μg of an anti-BST2 antibody (mAb 927) at 48 h and 24 h prior to PapMV ssRNA-VLP immunization. IFN-a production in serum and spleen was monitored by ELISA (VeriKine™ Mouse Interferon Alpha ELISA Kit; PBL InterferonSource) at 6, 12, 24 and 48 h post-immunization (FIG. 11A) or at 6 h after immunization (FIG. 11B).

The results are shown in FIG. 11 and indicate that IFN-α production can be effectively stimulated by PapMV ssRNA-VLPs when administered intravenously, and that this stimulation depends on MYD88, TLR7 and BST2⁺ cells.

### Intraperitoneal Administration

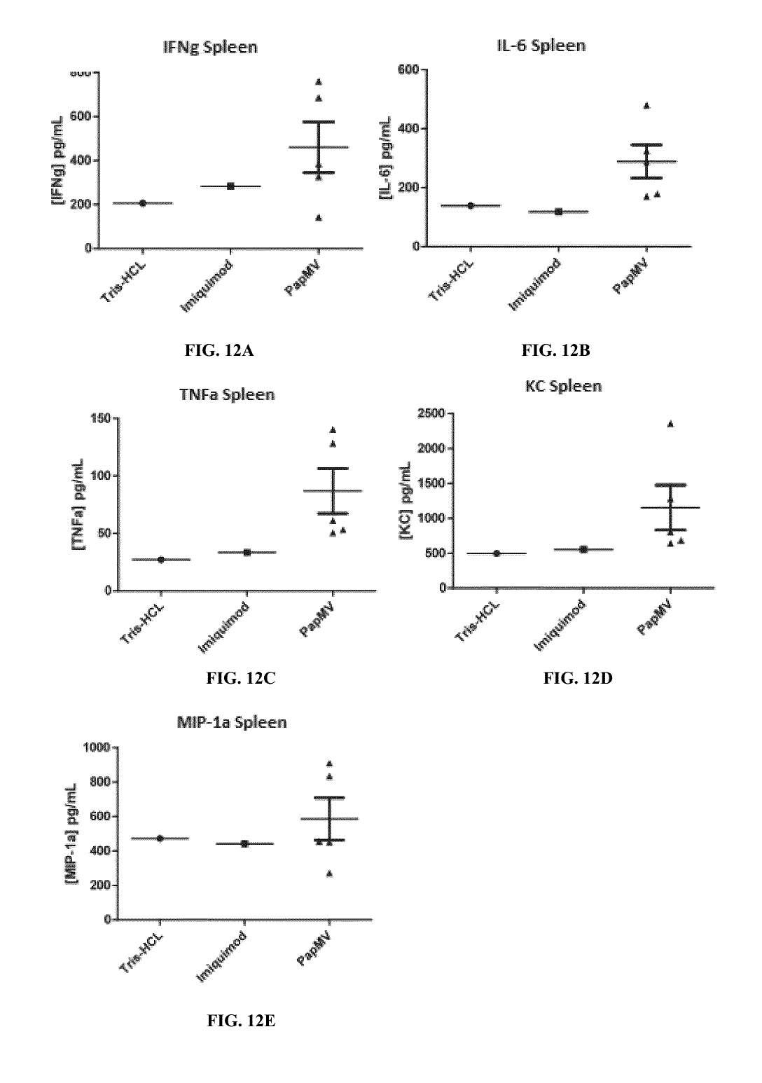

Experiment 1: Balb/C mice were injected i.p. with a volume of 200 μL containing either Tris-HCl buffer 10 mM pH8.0, 15 μg Imiquimod or 100 μg of PapMV ssRNA-VLPs. Six hours after injection, the spleen of the animal was collected by surgery and lysed. The lysate was filtered and centrifuged. The supernatants were analyzed by LUMINEX for the presence of (i) the cytokines: IFN-gamma (IFN-g), IL-6, TNF-alpha (TNF-a), (ii) keratinocyte chemoattractant (KC) and (iii) the chemokine MIP-1alpha (MIP-1a). The results are shown in FIG. 12.

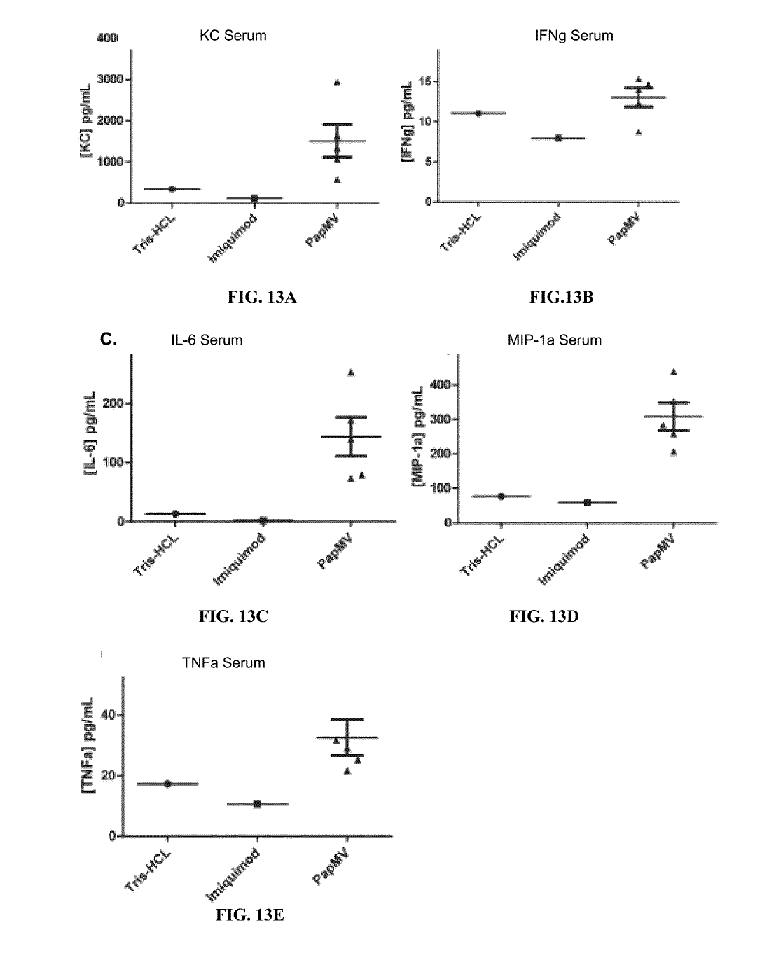

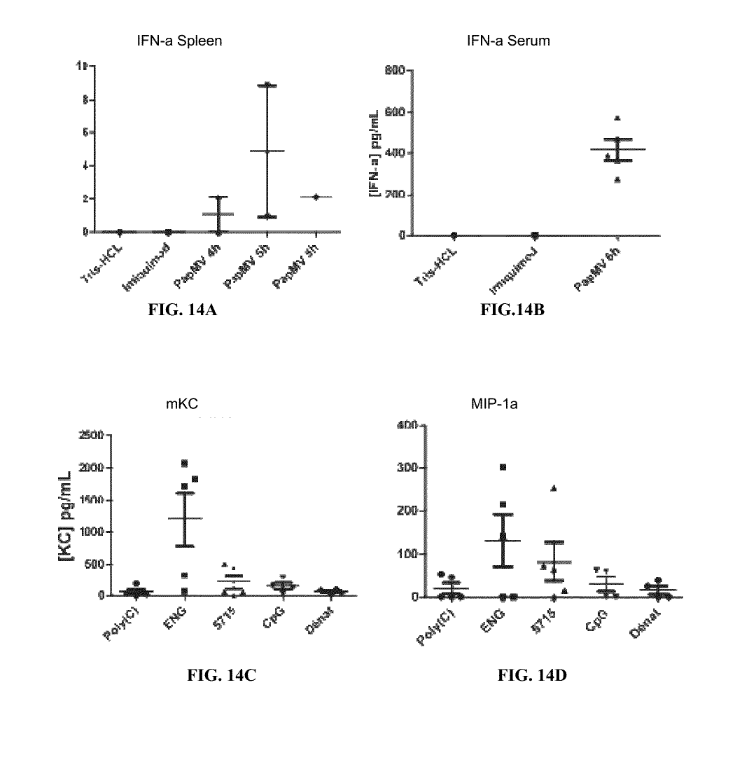

Blood was also collected during surgery when the spleen was extracted from each animal and the serum was also analyzed for the presence of cytokines and chemokines by LUMINEX. The results are shown in FIG. 13.

Experiment 2: Balb/C mice (2 per groups) were injected i.p. with a volume of 200 μL containing either the Tris-HCl buffer 10 mM pH8.0, 15 μg Imiquimod or 100 μg of PapMV ssRNA-VLPs. Four, five or six hours after injection, the spleen of the animal was collected by surgery and lysed. The lysate was filtered and centrifuged. The supernatants were analyzed by LUMINEX for the presence of interferon-α (IFN-α). The results are shown in FIG. 14(A).

Using a similar protocol as described for Experiment 1, serum was collected 6 hours after i.p. injection with either Tris-HCl pH8 10 mM, 15 μg Imiquimod or 100 μg PapMV ssRNA-VLPs and analyzed for the presence of interferon-α (IFN-α). The results are shown in FIG. 14(B).

Experiment 3: In order to validate the results of Experiments 1 and 2 above, a third experiment was conducted using PapMV ssRNA-VLPs produced in an “engineering run” (designated “ENG”), PapMV VLPs that were self-assembled with a polyC RNA rather than ssRNA, “lot 5715 PapMV VLPs,” CpG (50 μg) and PapMV ssRNA-VLPs denatured by heating at 60° C. for 30 min. (“Dénat”). The polyC RNA-VLPs, “lot 5715 PapMV VLPs” and denatured ssRNA-VLPs were included as negative controls. PolyC RNA-VLPs are known to have only a weak adjuvant property. “Lot 5715 PapMV VLPs” are VLPs that were oxidized during production resulting in aberrant self-assembly with the resulting VLPs being extremely short and exhibiting very poor immunogenicity and adjuvant activity. Heat treatment of the ssRNA-VLPs is known to destroy the structure of the particles, which is important for their immunomodulatory effects. The results are shown in FIG. 14(C) & (D).

In summary, the experiments in this Example show that innate immunity can efficiently be triggered through i.n, i.v. and i.p. injection of PapMV ssRNA-VLPs. FIG. 10 shows that innate immunity could be triggered in the lungs through intranasal immunization of PapMV ssRNA-VLPs as early as 6 hours after injection and several cytokines and chemokines, including MIP-1a, TNF-a, IL-6 and KC, were induced. FIG. 11 shows that PapMV ssRNA-VLPs were able to trigger innate immunity by i.v. injection. Using this route of immunization, secretion of IFN-a could be detected in the spleen and the serum of the immunized animals 6 hours after injection. FIGS. 12-14 show that PapMV ssRNA-VLPs administered i.p. efficiently trigger an innate immune response as early as 5 hours after injection.

The anti-cancer activity of the PapMV ssRNA-VLPs is predicted to be due to its ability to trigger innate immunity. Based on the above results, it is anticipated that the i.n., i.p. and i.v. routes of immunization can be used to induce innate immunity in patients suffering from cancer. This, in turn, will improve the immune response directed to the cancer cells and lead to an improvement in the disease state of the patient.

### Example 4

### PapMV ssRNA-VLPs Inhibit Tumour Growth and Potentiate the Anti-Cancer Effects of Dendritic Cells In Vivo #2

The experiments investigating the effect of PapMV ssRNA-VLPs in the anti-tumour response against subcutaneous melanoma described in Example 1 were repeated with the following modifications. 5×10⁵ B16-OVA cells were injected subcutaneously (s.c.) into the mice. Treatments were administered at day 7, 12 and 17 post tumor cell inoculation. Some mice were euthanized 6 h after the second PapMV ssRNA-VLPs injection for cytokine and chemokine analysis. Others were euthanized at day 15 or 16 post tumour inoculation for immune response analysis by flow cytometry. Finally, for the tumour growth and survival monitoring, mice were euthanized when the tumour reached a diameter of 17 mm.

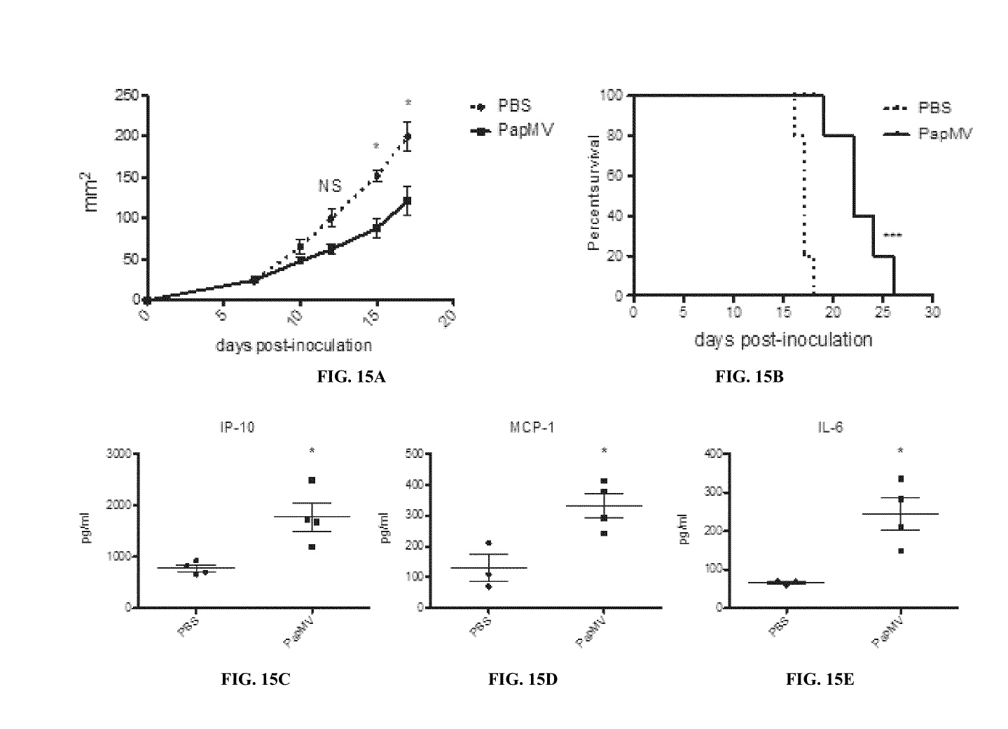

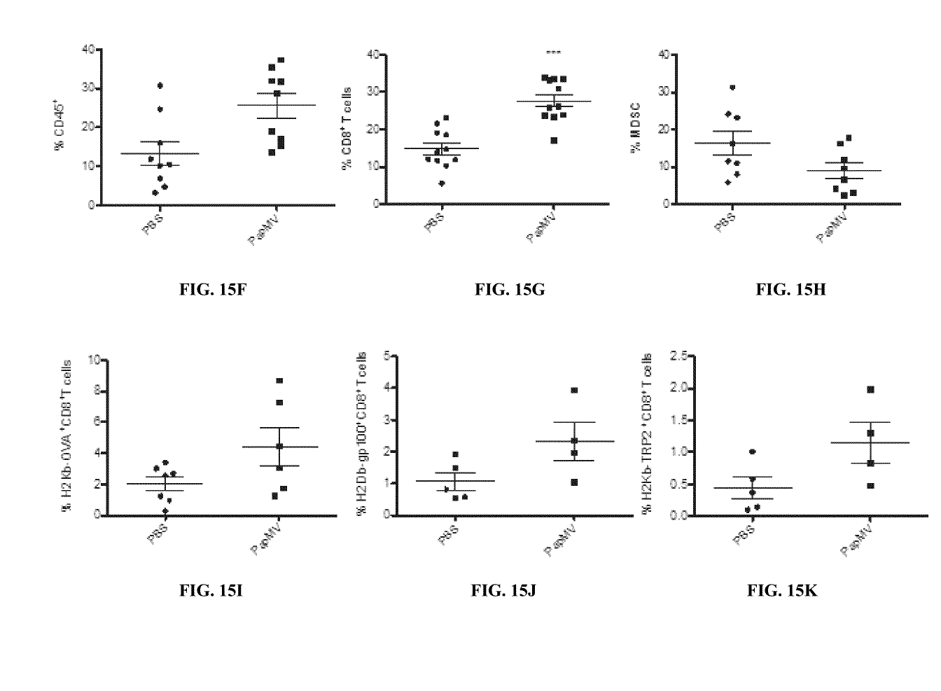

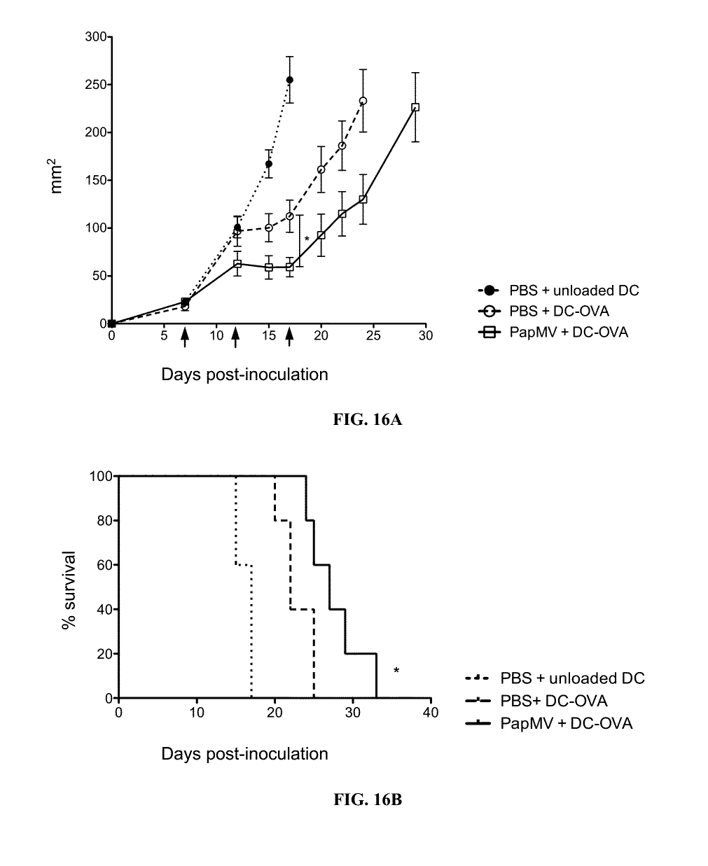

The results are shown in FIGS. 15 and 16 and confirm that intratumoral administration of PapMV ssRNA-VLPs alone decreased the growth rate of B16-OVA (FIG. 15A). In addition, this treatment was observed to increase survival of the mice (FIG. 15B). When PapMV ssRNA-VLPs was injected i.t. at day 7 and 12 post tumour cells inoculation, an increased immune cell (CD45+) infiltration into the tumour at day 15 was observed (FIG. 15F). In addition, proportions of different types of immune cells appeared to be changed with the treatment. In particular, an increased proportion of CD8+ T cells and a decrease in myeloid-derived suppressor cells (MDSC) was observed (FIG. 15G,H). A higher proportion of tumour-specific CD8+ T cells was also observed (FIG. 15I-K).

PapMV ssRNA-VLPs also increased the therapeutic effect of BMDC-OVA treatment, as well as survival of the mice (FIG. 16). Complement depletion (20 μg cobra venom factor, i.p.) in these situations did not increase the beneficial effect of the PapMV ssRNA-VLPs treatment.

### Example 5

### Effect of PapMV ssRNA-VLPs in Combination with High-Dose Cyclophosphamide on Tumour Growth

An initial experiment conducted using PapMV ssRNA-VLPs (administered i.v.) in combination with 250 mg/mL cyclophosphamide (CTX) showed no difference in effect between the combination and CTX alone. Two subsequent experiments were conducted using a lower dose of CTX (100 mg/kg). This dose is still generally considered as high-dose CTX (see, for example, Veltman et al., 2010, J. Biomedicine Biotechnol., Article ID 798467).

### Experiment 1

Design: 4 groups of 10 female C57BL6 mice (6-8 weeks old) were injected on day 0 with 6×10⁵ B16 melanoma cells in PBS. On day 9, half the mice were injected IV with 100 μg PapMV ssRNA-VLPs and the other half were injected 2004 Tris-HCL 10 mM. On day 11, tumors were measured and half the mice were injected intraperitoneally with 2 mg (100 mg/kg) of cyclophosphamide (CTX) and the other half with 200 μL of phosphate buffered saline. Tumours were measured every other day thereafter. On day 14 and 18 post-tumour injections, one group that received CTX and one group that received PBS were intravenously administered 100 μg PapMV ssRNA-VLPs. The other mice received Tris-HCL 10 mM as a control. On day 25 the protocol was terminated.

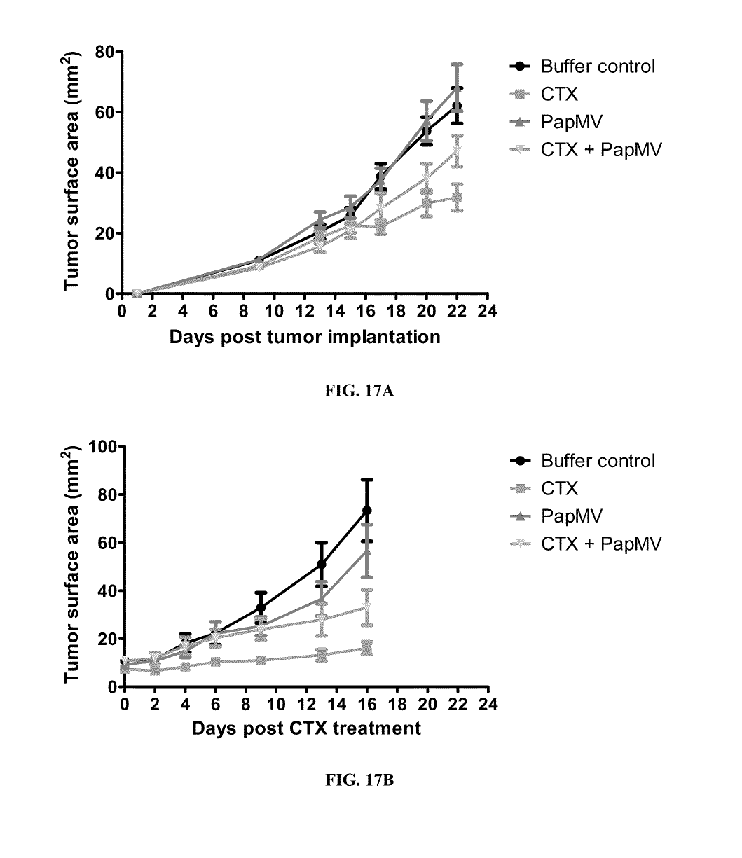

Results: The results are shown in FIG. 17A. As expected, intravenous administration of PapMV ssRNA-VLPs alone does not slow or accelerate tumour growth compared to buffer control. In combination with CTX, PapMV ssRNA-VLPs administered IV were less effective than CTX alone.

### Experiment 2

Design: 4 groups of 10 female C57BL6 mice (6-8 weeks old) were injected on day −9 with 6×10⁵ B16 melanoma cells in PBS. On day 0, tumors were measured and half the mice were injected intraperitoneally with 2 mg (100 mg/kg) of cyclophosphamide (CTX) and the other half with 200 μL of phosphate buffered saline (PBS). Tumours were measured every other day thereafter. On day 2, 7 and 12 post-CTX treatment, one group that received CTX and one group that received PBS were injected with 100 μg PapMV ssRNA-VLPs intratumorally (IT). The other mice received Tris-HCL 10 mM as a control.

Results: The results are shown in FIG. 17B. PapMV ssRNA-VLPs administered IT showed a tendency toward a slower tumour growth compared to buffer control. The combination of high dose CTX with the PapMV ssRNA-VLPs improved this effect further. On the other hand, the group treated with CTX alone shows a very slow tumour growth. This may in part be due to the fact that the tumours in the CTX group were smaller at the onset of treatment compared to tumours of the CTX+PapMV ssRNA-VLPs group. The experiment confirmed that PapMV ssRNA-VLPs delivered inside the tumours has some effect and that this effect can be improved by the combination with chemotherapy.

Both the above experiments indicated that the combination of PapMV ssRNA-VLPs with high dose CTX does not result in an improvement over the anti-tumour effects of CTX alone. This result is not unexpected as the known effects of high dose CTX on T cell subsets in treated animals (see, for example, Motoyoshi et al., 2006, ibid.) impairs the efficacy of the immune system in general which consequently impacts the efficacy of the PapMV ssRNA-VLPs in increasing the anti-tumour response.

It is anticipated that combination of PapMV ssRNA-VLPs with low dose CTX (i.e. less than 100 mg/kg) will show an improvement in the anti-tumour effects of low dose CTX, which has a much lesser impact on T cell populations. In particular, doses of 10 mg/kg of CTX in mice have been shown to be effective for stimulating cell-mediated immunity (Otterness& Chang, 1976, Clin. Exp. Immunol., 26:346-354) and may thus be useful. Combination of PapMV ssRNA-VLPs with other chemotherapeutic agents that have a different mechanism of action to cyclophosphamide are also expected to synergize better with PapMV IT to suppress tumour growth.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A composition comprising papaya mosaic virus (PapMV) or PapMV virus-like particles (VLPs) comprising ssRNA for use in the treatment of cancer in a subject in need thereof.

2. The composition according to claim 1, wherein the composition is for administration intratumorally.

3. The composition according to claim 1 or 2, wherein the composition is for use in combination with another cancer therapy.

4. The composition according to any one of claims 1 to 3, wherein the treatment comprises inhibiting growth of the cancer.

5. The composition according to claim 3, wherein the treatment comprises inhibiting metastasis of the cancer.

6. The composition according to claim 3 or 5, wherein the cancer therapy comprises one or more of radiotherapy, chemotherapy and immunotherapy.

7. The composition according to claim 3 or 5, wherein the composition is for use in combination with an immunotherapeutic.

8. The composition according to claim 7, wherein the immunotherapeutic is a cell-based cancer immunotherapeutic.

9. The composition according to claim 8, wherein the cell-based cancer immunotherapeutic is a dendritic cell-based immunotherapeutic.

10. A composition comprising papaya mosaic virus (PapMV) or PapMV virus-like particles (VLPs) comprising ssRNA for use to improve a cancer immunotherapy in treatment of cancer in a subject in need thereof.

11. The composition according to claim 10, wherein the composition is for administration to the subject prior to administration of the cancer immunotherapy.

12. The composition according to claim 10 or 11, wherein the cancer immunotherapy comprises dendritic cells loaded with a cancer specific antigen.

13. The composition according to any one of claims 10 to 12, wherein the treatment comprises inhibiting growth of the cancer.

14. The composition according to any one of claims 10 to 13, wherein the treatment comprises inhibiting metastasis of the cancer.

15. The composition according to any one of claims 1 to 14, wherein the composition comprises the PapMV VLPs.

16. A composition comprising papaya mosaic virus (PapMV) virus-like particles (VLPs) comprising ssRNA for use in the treatment of cancer in a subject in need thereof, wherein the composition is for intratumoral administration and wherein the composition inhibits growth of the cancer.

17. A composition comprising papaya mosaic virus (PapMV) virus-like particles (VLPs) comprising ssRNA for use to improve a dendritic cell-based immunotherapy in treatment of cancer in a subject in need thereof.

18. The composition according to claim 17, wherein the composition is for administration to the subject prior to administration of the dendritic cell-based immunotherapy.

19. The composition according to any one of claims 1 to 18, wherein the cancer is a solid tumour.

20. The composition according to any one of claims 1 to 15, wherein the cancer is bladder cancer, breast cancer, colon cancer, kidney cancer, lung cancer, prostate cancer, leukemia, lymphoma, multiple myeloma or melanoma.

21. The composition according to any one of claims 1 to 20, wherein the ssRNA comprised by the PapMV VLPs is between about 50 nucleotides and about 5000 nucleotides in length.

22. The composition according to any one of claims 1 to 20, wherein the ssRNA comprised by the PapMV VLPs is between about 1000 and about 3000 nucleotides in length.

23. The composition according to any one of claims 1 to 22, wherein the ssRNA comprised by the PapMV VLPs is synthetic ssRNA.

24. The composition according to claim 23, wherein the synthetic ssRNA does not include any AUG codons.

25. The composition according to claim 23 or 24, wherein the synthetic ssRNA comprises a sequence corresponding to nucleotides 17 to 54 of SEQ ID NO:1.

26. The composition according to claim 23 or 24, wherein the synthetic ssRNA comprises a sequence corresponding to the nucleic acid sequence as set forth in SEQ ID NO:1 or 6, or a fragment thereof.

27. The composition according to any one of claims 1 to 26, wherein the subject is a human.

28. A method of treating cancer in a subject comprising administering to the subject an effective amount of a composition comprising papaya mosaic virus (PapMV) or PapMV virus-like particles (VLPs) comprising ssRNA.

29. The method according to claim 28, wherein the composition is administered intratumorally.

30. The method according to claim 28 or 29, wherein the composition is administered in combination with another cancer therapy.

31. The method according to any one of claims 28 to 30, wherein the treatment comprises inhibiting growth of the cancer.

32. The method according to claim 30, wherein the treatment comprises inhibiting metastasis of the cancer.

33. The method according to claim 30 or 32, wherein the cancer therapy comprises one or more of radiotherapy, chemotherapy and immunotherapy.

34. The composition according to claim 30 or 32, wherein the composition is administered in combination with an immunotherapeutic.

35. The method according to claim 34, wherein the immunotherapeutic is a cell-based cancer immunotherapeutic.

36. The method according to claim 35, wherein the cell-based cancer immunotherapeutic is a dendritic cell-based immunotherapeutic.

37. A method of improving a cancer immunotherapy in treating cancer in a subject comprising administering to the subject an effective amount of a composition comprising papaya mosaic virus (PapMV) or PapMV virus-like particles (VLPs) comprising ssRNA.

38. The method according to claim 37, wherein the composition is administered to the subject prior to administration of the cancer immunotherapy.

39. The method according to claim 37 or 38, wherein the cancer immunotherapy comprises dendritic cells loaded with a cancer specific antigen.

40. The method according to any one of claims 37 to 39, wherein the treatment comprises inhibiting growth of the cancer.

41. The method according to any one of claims 37 to 39, wherein the treatment comprises inhibiting metastasis of the cancer.

42. The method according to any one of claims 28 to 41, wherein the composition comprises the PapMV VLPs.

43. A method of treating cancer in a subject comprising administering to the subject an effective amount of a composition comprising papaya mosaic virus (PapMV) virus-like particles (VLPs) comprising ssRNA, wherein the composition is administered intratumorally and wherein the composition inhibits growth of the cancer.

44. A method of improving a dendritic cell-based immunotherapy in treating cancer in a subject comprising administering to the subject an effective amount of a composition comprising papaya mosaic virus (PapMV) virus-like particles (VLPs) comprising ssRNA.

45. The method according to claim 44, wherein the composition is administered to the subject prior to administration of the dendritic cell-based immunotherapy.

46. The method according to any one of claims 28 to 45, wherein the cancer is a solid tumour.

47. The method according to any one of claims 28 to 45, wherein the cancer is bladder cancer, breast cancer, colon cancer, kidney cancer, lung cancer, prostate cancer, leukemia, lymphoma, multiple myeloma or melanoma.

48. The method according to any one of claims 28 to 47, wherein the ssRNA comprised by the PapMV VLPs is between about 50 nucleotides and about 5000 nucleotides in length.

49. The method according to any one of claims 28 to 47, wherein the ssRNA comprised by the PapMV VLPs is between about 1000 and about 3000 nucleotides in length.

50. The method according to any one of claims 28 to 49, wherein the ssRNA comprised by the PapMV VLPs is synthetic ssRNA.

51. The method according to claim 50, wherein the synthetic ssRNA does not include any AUG codons.

52. The method according to claim 50 or 51, wherein the synthetic ssRNA comprises a sequence corresponding to nucleotides 17 to 54 of SEQ ID NO:1.

53. The method according to claim 50 or 51, wherein the synthetic ssRNA comprises a sequence corresponding to the nucleic acid sequence as set forth in SEQ ID NO:1 or 6, or a fragment thereof.

54. The method according to any one of claims 29 to 53, wherein the subject is a human.
